(19) United States
(12) Patent Application Publication
(10) Pub. No.: US 2001/0044134 A1
(43) Pub. Date: Nov. 22, 2001

(54) Novel secreted polypeptide zsig87

(76) Inventor: Paul O. Sheppard, Granite Falls, WA (US)

Correspondence Address: Jennifer K. Johnson, J.D., ZymoGenetics, Inc., 1201 Eastlake Avenue East, Seattle, WA, 98102, US

(21) Appl. No.: 09/733,523

(22) Filed: Dec. 8, 2000

Related U.S. Application Data
(60) Provisional application No. 60/169,597, filed on Dec. 8, 1999.

(51) Int. Cl. C12Q 1/68; G01N 33/574; C07H 21/04; C12P 21/04; C07K 16/18; C07K 14/435

(52) U.S. Cl. 435/69.7; 435/6; 435/7.23; 435/325; 530/388.1; 530/350; 435/70.21; 536/23.5

(57) ABSTRACT

The present invention relates to polynucleotide and polypeptide molecules for zsig87, a novel secreted protein. The polynucleotides encoding zsig87, may, for example, be used to identify a region of the genome associated with human disease states. The present invention also includes methods for producing the protein, uses therefor and antibodies thereto.

### REFERENCE TO RELATED APPLICATION

This application is related to Provisional Application No. 60/169,597, filed on Dec. 8, 1999. Under 35 U.S.C. § 119(e)(1), this application claims benefit of said Provisional Application.

### BACKGROUND OF THE INVENTION

Hormones and polypeptide growth factors control proliferation and differentiation of cells of multicellular organisms. These diffusible molecules allow cells to communicate with each other and act in concert to regulate cells and form organs, and to repair and regenerate damaged tissue. Examples of hormones and growth factors include the steroid hormones (e.g. estrogen, testosterone), parathyroid hormone, follicle stimulating hormone, the interleukins, platelet derived growth factor (PDGF), epidermal growth factor (EGF), granulocyte-macrophage colony stimulating factor (GM-CSF), erythropoietin (EPO) and calcitonin.

Hormones and growth factors influence cellular metabolism by binding to proteins. These proteins may be integral membrane proteins that are linked to signaling pathways within the cell, such as second messenger systems. Other classes of proteins that hormones and growth factors influence are soluble molecules, such as the transcription factors.

Thus, there is a continuing need to discover new hormones, growth factors and the like. The in vivo activities of these cytokines illustrates the enormous clinical potential of, and need for, other cytokines, cytokine agonists, and cytokine antagonists.

The present invention provides such polypeptides for these and other uses that should be apparent to those skilled in the art from the teachings herein.

### DESCRIPTION OF THE INVENTION

Within one aspect, the present invention provides an isolated polynucleotide encoding a zsig87 polypeptide comprising a sequence of amino acid residues that is at least 90% identical to an amino acid sequence selected from the group consisting of: (a) the amino acid sequence as shown in SEQ ID NO:2 from amino acid number 27 (Arg) to amino acid number 56 (Cys); (b) the amino acid sequence as shown in SEQ ID NO:2 from amino acid number 16 (Glu) to amino acid number 84 (Pro); and (c) the amino acid sequence as shown in SEQ ID NO:2 from amino acid number 1 (Met) to amino acid number 84 (Pro), wherein the amino acid percent identity is determined using a FASTA program with ktup=1, gap opening penalty=10, gap extension penalty=1, and substitution matrix=BLOSUM62, with other parameters set as default. In one embodiment, the isolated polynucleotide disclosed above is selected from the group consisting of: (a) a polynucleotide sequence as shown in SEQ ID NO:1 from nucleotide 198 to nucleotide 287; (b) a polynucleotide sequence as shown in SEQ ID NO:1 from nucleotide 165 to nucleotide 371; (c) a polynucleotide sequence as shown in SEQ ID NO:1 from nucleotide 120 to nucleotide 371; and (c) a polynucleotide sequence complementary to (a) or (b). In another embodiment, the isolated polynucleotide disclosed above comprises nucleotide 1 to nucleotide 252 of SEQ ID NO:3. In another embodiment, the isolated polynucleotide disclosed above wherein the polynucleotide encodes a polypeptide comprising a sequence of amino acid residues selected from the group consisting of: (a) the amino acid sequence as shown in SEQ ID NO:2 from amino acid number 27 (Arg) to amino acid number 56 (Cys); (b) the amino acid sequence as shown in SEQ ID NO:2 from amino acid number 16 (Glu) to amino acid number 84 (Pro); and (c) the amino acid sequence as shown in SEQ ID NO:2 from amino acid number 1 (Met) to amino acid number 84 (Pro). In one embodiment, the isolated polynucleotide disclosed above, wherein the polynucleotide encodes a polypeptide that further comprises a cysteine motif spaced apart from N-terminus to C-terminus in a configuration represented by C-{4}-G-C-(K/R)-{2}-C-{9/13}-C-{5}-C-C, where “C” is cysteine, “G” is Glycine, “(K/R)” is Lysine or Arginine, “{#}” denotes the number of amino acid residues between the above amino acids, “{9/13}” denotes that the number of amino acid residues between the above amino acids is 9 or 13.

Within a second aspect, the present invention provides an expression vector comprising the following operably linked elements: a transcription promoter; a DNA segment encoding a zsig87 polypeptide comprising an amino acid sequence as shown in SEQ ID NO:2 from amino acid number 16 (Glu) to amino acid number 84 (Pro); and a transcription terminator.

Within a third aspect, the present invention provides an expression vector as disclosed above, further comprising a secretory signal sequence operably linked to the DNA segment.

Within a fourth aspect, the present invention provides a cultured cell into which has been introduced an expression vector as disclosed above, wherein the cell expresses a polypeptide encoded by the DNA segment.

Within another aspect, the present invention provides a DNA construct encoding a fusion protein, the DNA construct comprising: a first DNA segment encoding a polypeptide comprising a sequence of amino acid residues selected from the group consisting of: (a) the amino acid sequence of SEQ ID NO: 2 from residue number 1 (Met) to amino acid number 15 (Glu); (a) the amino acid sequence of SEQ ID NO: 2 from residue number 27 (Arg) to amino acid number 84 (Pro); and (b) the amino acid sequence of SEQ ID NO: 2 from residue number 16 (Glu) to amino acid number 84 (Pro); and at least one other DNA segment encoding an additional polypeptide, wherein the first and other DNA segments are connected in-frame; and encode the fusion protein.

Within another aspect, the present invention provides a fusion protein produced by a method comprising: culturing a host cell into which has been introduced a vector comprising the following operably linked elements: (a) a transcriptional promoter; (b) a DNA construct encoding a fusion protein as disclosed above; and (c) a transcriptional terminator; and recovering the protein encoded by the DNA segment.

Within another aspect, the present invention provides an isolated polypeptide comprising a sequence of amino acid residues that is at least 90% identical to an amino acid sequence selected from the group consisting of: (athe amino acid sequence as shown in SEQ ID NO:2 from amino acid number 27 (Arg) to amino acid number 56 (Cys); (b) the amino acid sequence as shown in SEQ ID NO:2 from amino acid number 16 (Glu) to amino acid number 84 (Pro); and (c) the amino acid sequence as shown in SEQ ID NO:2 from amino acid number 1 (Met) to amino acid number 84 (Pro), wherein the amino acid percent identity is determined using a FASTA program with ktup=1, gap opening penalty=10, gap extension penalty=1, and substitution matrix=BLOSUM62, with other parameters set as default. In one embodiment, the isolated polypeptide disclosed above further comprises a cysteine motif spaced apart from N-terminus to C-terminus in a configuration represented by C-{4}-G-C-(K/R)-{2}-C-{9/13}-C-{5}-C-C, where “C” is cysteine, “G” is Glycine, “(K/R)” is Lysine or Arginine, “{#}” denotes the number of amino acid residues between the above amino acids, “{9/13}” denotes that the number of amino acid residues between the above amino acids is 9 or 13. In another embodiment, the isolated polypeptide disclosed above comprises a sequence of amino acid residues selected from the group consisting of: (a) the amino acid sequence as shown in SEQ ID NO:2 from amino acid number 27 (Arg) to amino acid number 56 (Cys); (b) the amino acid sequence as shown in SEQ ID NO:2 from amino acid number 16 (Glu) to amino acid number 84 (Pro); and (c) the amino acid sequence as shown in SEQ ID NO:2 from amino acid number 1 (Met) to amino acid number 84 (Pro).

Within another aspect, the present invention provides a method of producing a zsig87 polypeptide comprising: culturing a cell as disclosed above; and isolating the zsig87 polypeptide produced by the cell.

Within another aspect, the present invention provides a method of detecting, in a test sample, the presence of a modulator of zsig87 protein activity, comprising: transfecting a zsig87-responsive cell, with a reporter gene construct that is responsive to a zsig87-stimulated cellular pathway; and producing a zsig87 polypeptide by the method as disclosed above; and adding the zsig87 polypeptide to the cell, in the presence and absence of a test sample; and comparing levels of response to the zsig87 polypeptide, in the presence and absence of the test sample, by a biological or biochemical assay; and determining from the comparison, the presence of the modulator of zsig87 activity in the test sample.

Within another aspect, the present invention provides a method of producing an antibody to zsig87 polypeptide comprising the following steps in order: inoculating an animal with a polypeptide selected from the group consisting of: (a) a polypeptide consisting of 9 to 57 amino acids, wherein the polypeptide has a contiguous sequence of amino acids as shown within SEQ ID NO:2 from amino acid number 16 (Glu) to amino acid number 84 (Pro); (b) a polypeptide as disclosed above; (c) a polypeptide comprising the amino acid sequence of SEQ ID NO: 2 from residue number 16 (Glu) to amino acid number 84 (Pro); (d) a polypeptide comprising the amino acid sequence of SEQ ID NO: 2 from residue number 27 (Arg) to amino acid number 56 (Cys); (e) a polypeptide comprising of the amino acid sequence of SEQ ID NO: 2 from residue number 57 (Leu) to amino acid number 84 (Pro); (f) a polypeptide consisting of amino acid number 39 (Cys) to amino acid number 45 (Pro) of SEQ ID NO:2; (g) a polypeptide consisting of amino acid number 41 (Lys) to amino acid 47 (Leu) of SEQ ID NO:2; (h) a polypeptide consisting of amino acid number 68 (Lys) to amino acid number 73 (Asp) of SEQ ID NO:2; (i) a polypeptide consisting of amino acid number 77 (Glu) to amino acid number 82 (Arg) of SEQ ID NO:2; and (j) a polypeptide consisting of amino acid number 68 (Lys) to amino acid number 82 (Arg) of SEQ ID NO:2; and wherein the polypeptide elicits an immune response in the animal to produce the antibody; and isolating the antibody from the animal.

Within another aspect, the present invention provides an antibody produced by the method as disclosed above, which binds to a zsig87 polypeptide. In one embodiment, the antibody disclosed above is a monoclonal antibody. Within another aspect, the present invention provides an antibody which specifically binds to a polypeptide as disclosed above.

Within another aspect, the present invention provides a method for detecting a genetic abnormality in a patient, comprising: obtaining a genetic sample from a patient; producing a first reaction product by incubating the genetic sample with a polynucleotide comprising at least 14 contiguous nucleotides of SEQ ID NO:1 or the complement of SEQ ID NO:1, under conditions wherein said polynucleotide will hybridize to complementary polynucleotide sequence; visualizing the first reaction product; and comparing said first reaction product to a control reaction product from a wild type patient, wherein a difference between said first reaction product and said control reaction product is indicative of a genetic abnormality in the patient.

These and other aspects of the invention will become evident upon reference to the following detailed description of the invention.

Prior to setting forth the invention in detail, it may be helpful to the understanding thereof to define the following terms:

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a polyhistidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag? peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “complements of a polynucleotide molecule” denotes a polynucleotide molecule having a complementary base sequence and reverse orientation as compared to a reference sequence. For example, the sequence 5′ ATGCACGGG 3′ is complementary to 5′ CCCGTGCAT 3′.

The term “contig” denotes a polynucleotide that has a contiguous stretch of identical or complementary sequence to another polynucleotide. Contiguous sequences are said to “overlap” a given stretch of polynucleotide sequence either in their entirety or along a partial stretch of the polynucleotide. For example, representative contigs to the polynucleotide sequence 5′-ATGGAGCTT-3′ are 5′-AGCTTgagt-3′ and 3′-tcgacTACC-5′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

“Probes and/or primers” as used herein can be RNA or DNA. DNA can be either cDNA or genomic DNA. Polynucleotide probes and primers are single or double-stranded DNA or RNA, generally synthetic oligonucleotides, but may be generated from cloned cDNA or genomic sequences or its complements. Analytical probes will generally be at least 20 nucleotides in length, although somewhat shorter probes (14-17 nucleotides) can be used. PCR primers are at least 5 nucleotides in length, preferably 15 or more nt, more preferably 20-30 nt. Short polynucleotides can be used when a small region of the gene is targeted for analysis. For gross analysis of genes, a polynucleotide probe may comprise an entire exon or more. Probes can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel DNA sequence that encodes a novel polypeptide. The polypeptide and corresponding polynucleotide were isolated from testis and mast cell libraries. The polypeptide has been designated zsig87.

The novel zsig87 polypeptides of the present invention were initially identified as polypeptides homologous to proteins having a secretory signal sequence and the corresponding cDNA was sequenced. The zsig87 nucleotide sequence is believed to encode the entire coding sequence of the predicted protein. Zsig87 may be a novel cell-cell signaling molecule, growth factor, cytokine, secreted extracellular matrix associated protein with growth factor hormone activity, or the like, and is a member a novel protein family.

The sequence of the zsig87 polypeptide was obtained from a single clone believed to contain its corresponding polynucleotide sequence. The clone was obtained from a testis library. Other libraries that might also be searched for such sequences include mast cells, peripheral blood leukocytes (PBLs), pancreas, liver, ovary, placenta, and the like.

The nucleotide sequence of a representative zsig87-encoding DNA is described in SEQ ID NO:1, and its deduced 84 amino acid sequence is described in SEQ ID NO:2. In its entirety, the zsig87 polypeptide (SEQ ID NO:2) represents a polypeptide segment containing an active polypeptide (residue 27 (Arg) to residue 56 (Cys) of SEQ ID NO:2) based on structural similarity with other secreted molecules (PAT_HSGSIG640 (WIPO publication WO 9907891); PSP_Y10813 (WIPO publication WO 9907891); PAT_HGSSIG917 (WIPO publication WO 9919339); PAT_HGSSIG1330 (WIPO publication WO 9931117); and PAT_GISIG166 (WIPO publication WO 9821332)) and some plant gibberellin- and temperature-regulated maturation factors (GAS3ARATH (Genbank Accession No. P46687); and LTCOR12 (Genbank Accession No. AF060569)). The domains and structural features of zsig87 are further described below.

Analysis of the zsig87 polypeptide encoded by the DNA sequence of SEQ ID NO:1 revealed an open reading frame encoding 84 amino acids (SEQ ID NO:2) comprising a predicted signal peptide of 15 amino acid residues (residue 1 (Met) to residue 15 (Glu) of SEQ ID NO:2), and a secreted polypeptide of 69 amino acids (residue 16 (Glu) to residue 84 (Pro) of SEQ ID NO:2). The zsig87 polypeptide contains several structural regions and motifs described below:

1) a small “N-terminal domain” comprising amino acid residues 16 (Glu) to 26 (Leu) of SEQ ID NO:2. Within the N-terminal domain is a potential dibasic protease cleavage site at Lys₂₂-Arg₂₃ of SEQ ID NO:2;

2) a “conserved domain” comprising amino acid residues 27 (Arg) to residue 56 (Cys) of SEQ ID NO:2. Within the conserved domain are conserved cysteines at positions 28, 35, 39, 49, 55, and 56 of SEQ ID NO:2. The cysteine pattern comprises a conserved motif, wherein the cysteines are spaced apart from N-terminus to C-terminus in a configuration represented by the following:

C-{4}-G-C-(K/R)-{2}-C-{9/13}-C-{5}-C-C

where “C” is cysteine,

“G” is Glycine,

“(K/R)” is Lysine or Arginine,

“{#}” denotes the number of amino acid residues between the above amino acids,

“{9/13}” denotes that the number of amino acid residues between the above amino acids is 9 or 13; and

3) a “C-terminal domain” comprising amino acid residues 57 (Leu) to 84 (Pro) of SEQ ID NO:2.

The presence of conserved and low variance motifs generally correlates with or defines important structural regions in proteins. Regions of low variance (e.g., hydrophobic clusters) are generally present in regions of structural importance (Sheppard, P. et al., Gene 150:163-167, 1994). Such regions of low variance often contain rare or infrequent amino acids, such as Tryptophan. The regions flanking and between such conserved and low variance motifs may be more variable, but are often functionally significant because they may relate to or define important structures and activities such as binding domains, biological and enzymatic activity, signal transduction, tissue localization domains and the like. In addition, the Cysteine residues that form disulfide bonds described above, can be important for structure or activity of zsig87 polypeptide.

The corresponding polynucleotides encoding the zsig87 polypeptide regions, domains, motifs, residues and sequences described above are as shown in SEQ ID NO:1.

The low degeneracy amino acids in zsig87 can be used as a tool to identify new family members. For instance, reverse transcription-polymerase chain reaction (RT-PCR) can be used to amplify sequences, encoding the low degeneracy motifs, from RNA obtained from a variety of tissue sources or cell lines. In particular, highly degenerate primers designed from the zsig87 sequences are useful for this purpose. In particular, degenerate oligonucleotide primers designed from the following zsig87 amino acid sequences are useful for this purpose:

a) CCLQSY (amino acid residues 59 to 64 of SEQ ID NO:2), corresponding to degenerate polynucleotides of SEQ ID NO:4 and their complement;

b) CRASCK (amino acid residues 35 to 40 of SEQ ID NO:2), corresponding to degenerate polynucleotides of SEQ ID NO:5 and their complement; and

c) FLAILL (amino acid residues 7 to 12 of SEQ ID NO:2), corresponding to degenerate polynucleotides of SEQ ID NO:6 and their complement.

The present invention also provides polynucleotide molecules, including DNA and RNA molecules, that encode the zsig87 polypeptides disclosed herein. Those skilled in the art will readily recognize that, in view of the degeneracy of the genetic code, considerable sequence variation is possible among these polynucleotide molecules. SEQ ID NO:3 is a degenerate DNA sequence that encompasses all DNAs that encode the zsig87 polypeptide of SEQ ID NO:2. Those skilled in the art will recognize that the degenerate sequence of SEQ ID NO:3 also provides all RNA sequences encoding SEQ ID NO:2 by substituting U for T. Thus, zsig87 polypeptide-encoding polynucleotides comprising nucleotide 1 to nucleotide 252 of SEQ ID NO:3 and their RNA equivalents are contemplated by the present invention. Table 1 sets forth the one-letter codes used within SEQ ID NO:3 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | | Nucleotide | |
|---|---|---|---|
| Base Code | Resolution | Base Code | Complement |
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ D NO:3, encompassing all possible codons for a given amino acid, are set forth in Table 2.

TABLE 2

| Three | One | | |
|---|---|---|---|
| Letter | Letter | | Degenerate |
| Code | Code | Synonymous Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid Threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:3 serves as a template for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention, isolated polynucleotides will hybridize to similar sized regions of SEQ ID NO:1, or to sequences complementary thereto, under stringent conditions. Within preferred embodiments of the invention the isolated polynucleotides will hybridize to similar sized regions of SEQ ID NO:1, other polynucleotide probes, primers, fragments and sequences recited herein or sequences complementary thereto. Polynucleotide hybridization is well known in the art and widely used for many applications, see for example, Sambrook et al., Molecular Cloning: A Laboratory Manual, Second Edition, Cold Spring Harbor, N.Y., 1989; Ausubel et al., eds., Current Protocols in Molecular Biology, John Wiley and Sons, Inc., NY, 1987; Berger and Kimmel, eds., Guide to Molecular Cloning Techniques, Methods in Enzymology, volume 152, 1987 and Wetmur, Crit. Rev. Biochem. Mol. Biol. 26:227-59, 1990. Polynucleotide hybridization exploits the ability of single stranded complementary sequences to form a double helix hybrid. Such hybrids include DNA-DNA, RNA-RNA and DNA-RNA.

Hybridization will occur between sequences which contain some degree of complementarity. Hybrids can tolerate mismatched base pairs in the double helix, but the stability of the hybrid is influenced by the degree of mismatch. The Tₘ of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases. Stringent hybridization conditions encompass temperatures of about 5-25° C. below the thermal melting point (Tₘ) of the hybrid and a hybridization buffer having up to 1 M Na⁺. Higher degrees of stringency at lower temperatures can be achieved with the addition of formamide which reduces the Tₘ of the hybrid about 1° C. for each 1% formamide in the buffer solution. Generally, such stringent conditions encompass temperatures of 20-70° C. and a hybridization buffer containing up to 6×SSC and 0-50% formamide. A higher degree of stringency can be achieved at temperatures of from 40-70° C. with a hybridization buffer having up to 4×SSC and from 0-50% formamide. Highly stringent conditions typically encompass temperatures of 42-70° C. with a hybridization buffer having up to 1×SSC and 0-50% formamide. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes.

The above conditions are meant to serve as a guide and it is well within the abilities of one skilled in the art to adapt these conditions for use with a particular polypeptide hybrid. The Tₘ for a specific target sequence is the temperature (under defined conditions) at which 50% of the target sequence will hybridize to a perfectly matched probe sequence. Those conditions which influence the Tₘ include, the size and base pair content of the polynucleotide probe, the ionic strength of the hybridization solution, and the presence of destabilizing agents in the hybridization solution. Numerous equations for calculating Tₘ are known in the art, see for example (Sambrook et al., ibid.; Ausubel et al., ibid.; Berger and Kimmel, ibid. and Wetmur, ibid.) and are specific for DNA, RNA and DNA-RNA hybrids and polynucleotide probe sequences of varying length. Sequence analysis software such as Oligo 4.0 (publicly available shareware) and Primer Premier (PREMIER Biosoft International, Palo Alto, Calif.) as well as sites on the Internet, are available tools for analyzing a given sequence and calculating Tₘ based on user defined criteria. Such programs can also analyze a given sequence under defined conditions and suggest suitable probe sequences. Typically, hybridization of longer polynucleotide sequences, >50 bp, is done at temperatures of about 20-25° C. below the calculated Tm. For smaller probes, <50 bp, hybridization is typically carried out at the Tₘ or 5-10° C. below. This allows for the maximum rate of hybridization for DNA-DNA and DNA-RNA hybrids.

The length of the polynucleotide sequence influences the rate and stability of hybrid formation. Smaller probe sequences, <50 bp, come to equilibrium with complementary sequences rapidly, but may form less stable hybrids. Incubation times of anywhere from minutes to hours can be used to achieve hybrid formation. Longer probe sequences come to equilibrium more slowly, but form more stable complexes even at lower temperatures. Incubations are allowed to proceed overnight or longer. Generally, incubations are carried out for a period equal to three times the calculated Cot time. Cot time, the time it takes for the polynucleotide sequences to reassociate, can be calculated for a particular sequence by methods known in the art.

The base pair composition of polynucleotide sequence will effect the thermal stability of the hybrid complex, thereby influencing the choice of hybridization temperature and the ionic strength of the hybridization buffer. A-T pairs are less stable than G-C pairs in aqueous solutions containing NaCl. Therefore, the higher the G-C content, the more stable the hybrid. Even distribution of G and C residues within the sequence also contribute positively to hybrid stability. Base pair composition can be manipulated to alter the Tₘ of a given sequence, for example, 5-methyldeoxycytidine can be substituted for deoxycytidine and 5-bromodeoxuridine can be substituted for thymidine to increase the Tₘ. 7-deazo-2′-deoxyguanosine can be substituted for guanosine to reduce dependence on Tₘ.

Ionic concentration of the hybridization buffer also effects the stability of the hybrid. Hybridization buffers generally contain blocking agents such as Denhardt's solution (Sigma Chemical Co., St. Louis, Mo.), denatured salmon sperm DNA, tRNA, milk powders (BLOTTO), heparin or SDS, and a Na⁺ source, such as SSC (1×SSC: 0.15 M NaCl, 15 mM sodium citrate) or SSPE (1×SSPE: 1.8 M NaCl, 10 mM NaH₂PO₄, 1 mM EDTA, pH 7.7). By decreasing the ionic concentration of the buffer, the stability of the hybrid is increased. Typically, hybridization buffers contain from between 10 mM-1 M Na⁺. Premixed hybridization solutions are also available from commercial sources such as Clontech Laboratories (Palo Alto, Calif.) and Promega Corporation (Madison, Wis.) for use according to manufacturer's instruction. Addition of destabilizing or denaturing agents such as formamide, tetralkylammonium salts, guanidinium cations or thiocyanate cations to the hybridization solution will alter the Tₘ of a hybrid. Typically, formamide is used at a concentration of up to 50% to allow incubations to be carried out at more convenient and lower temperatures. Formamide also acts to reduce non-specific background when using RNA probes.

As previously noted, the isolated polynucleotides of the present invention include DNA and RNA. Methods for preparing DNA and RNA are well known in the art. In general, RNA is isolated from a tissue or cell that produces large amounts of zsig87 RNA. Such tissues and cells are identified by Northern blotting (Thomas, Proc. Natl. Acad. Sci. USA 77:5201, 1980), and include islets of Langerhans, pancreas, tissues of endocrine origin, prostate, uterus, and testis, including whole testis tissue extracts or testicular cells, such as Sertoli cells, Leydig cells, spermatogonia, or epididymis, cells from vas deferens, and cervical cells, and the like although DNA can also be prepared using RNA from other tissues or isolated as genomic DNA. Total RNA can be prepared using guanidinium isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding zsig87 polypeptides are then identified and isolated by, for example, hybridization or PCR.

A full-length clone encoding zsig87 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to zsig87, receptor fragments, or other specific binding partners.

The polynucleotides of the present invention can also be synthesized using DNA synthesis machines. Currently the method of choice is the phosphoramidite method. If chemically synthesized double stranded DNA is required for an application such as the synthesis of a gene or a gene fragment, then each complementary strand is made separately. The production of short polynucleotides (60 to 80 bp) is technically straightforward and can be accomplished by synthesizing the complementary strands and then annealing them. However, for producing longer polynucleotides (longer than about 300 bp), special strategies are usually employed, because the coupling efficiency of each cycle during chemical DNA synthesis is seldom 100%. To overcome this problem, synthetic genes (double-stranded) are assembled in modular form from single-stranded fragments that are from 20 to 100 nucleotides in length.

One method for building a synthetic gene requires the initial production of a set of overlapping, complementary oligonucleotides, each of which is between 20 to 60 nucleotides long. Each internal section of the gene has complementary 3′ and 5′ terminal extensions designed to base pair precisely with an adjacent section. Thus, after the gene is assembled, process is completed by sealing the nicks along the backbones of the two strands with T4 DNA ligase. In addition to the protein coding sequence, synthetic genes can be designed with terminal sequences that facilitate insertion into a restriction endonuclease site of a cloning vector. An alternative way to prepare a full-length gene is to synthesize a specified set of overlapping oligonucleotides (40 to 100 nucleotides). After the 3′ and 5′ short overlapping complementary regions are annealed, large gaps still remain, but the short base-paired regions are both long enough and stable enough to hold the structure together. The gaps are filled and the DNA duplex is completed via enzymatic DNA synthesis by E. coli DNA polymerase I. After the enzymatic synthesis is completed, the nicks are sealed. Double-stranded constructs are sequentially linked to one another to form the entire gene sequence which is verified by DNA sequence analysis. See Glick and Pasternak, Molecular Biotechnology, Principles & Applications of Recombinant DNA, (ASM Press, Washington, D.C. 1994); Itakura et al., Annu. Rev. Biochem. 53: 323-56, 1984 and Climie et al., Proc. Natl. Acad. Sci. USA 87:633-7, 1990.

Zsig87 polynucleotide sequences disclosed herein can also be used as probes or primers to clone 5′ non-coding regions of a zsig87 gene. In view of the tissue-specific expression observed for zsig87 by Northern blotting, this gene region is expected to provide for testis and mast cell-specific expression. Promoter elements from a zsig87 gene could thus be used to direct the tissue-specific expression of heterologous genes in, for example, transgenic animals or patients treated with gene therapy. Cloning of 5′ flanking sequences also facilitates production of zsig87 proteins by “gene activation” as disclosed in U.S. Pat. No. 5,641,670. Briefly, expression of an endogenous zsig87 gene in a cell is altered by introducing into the zsig87 locus a DNA construct comprising at least a targeting sequence, a regulatory sequence, an exon, and an unpaired splice donor site. The targeting sequence is a zsig87 5′ non-coding sequence that permits homologous recombination of the construct with the endogenous zsig87 locus, whereby the sequences within the construct become operably linked with the endogenous zsig87 coding sequence. In this way, an endogenous zsig87 promoter can be replaced or supplemented with other regulatory sequences to provide enhanced, tissue-specific, or otherwise regulated expression.

The present invention further provides counterpart polypeptides and polynucleotides from other species (orthologs). These species include, but are not limited to mammalian, avian, amphibian, reptile, fish, insect and other vertebrate and invertebrate species. Of particular interest are zsig87 polypeptides from other mammalian species, including murine, porcine, ovine, bovine, canine, feline, equine, and other primate polypeptides. Orthologs of human zsig87 can be cloned using information and compositions provided by the present invention in combination with conventional cloning techniques. For example, a cDNA can be cloned using mRNA obtained from a tissue or cell type that expresses zsig87 as disclosed herein. Suitable sources of mRNA can be identified by probing Northern blots with probes designed from the sequences disclosed herein. A library is then prepared from mRNA of a positive tissue or cell line. A zsig87-encoding cDNA can then be isolated by a variety of methods, such as by probing with a complete or partial human cDNA or with one or more sets of degenerate probes based on the disclosed sequences. A cDNA can also be cloned using the polymerase chain reaction (PCR) (Mullis, U.S. Pat. No. 4,683,202), using primers designed from the representative human zsig87 sequence disclosed herein. Within an additional method, the cDNA library can be used to transform or transfect host cells, and expression of the cDNA of interest can be detected with an antibody to zsig87 polypeptide. Similar techniques can also be applied to the isolation of genomic clones.

Those skilled in the art will recognize that the sequence disclosed in SEQ ID NO:1 represents a single allele of human zsig87 and that allelic variation and alternative splicing are expected to occur. Allelic variants of this sequence can be cloned by probing cDNA or genomic libraries from different individuals according to standard procedures. Allelic variants of the DNA sequence shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the zsig87 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The present invention also provides isolated zsig87 polypeptides that are substantially similar to the polypeptides of SEQ ID NO:2 and their orthologs. The term “substantially similar” is used herein to denote polypeptides having 70%, preferably 75%, more preferably at least 80%, sequence identity to the sequences shown in SEQ ID NO:2 or their orthologs. Such polypeptides will more preferably be at least 90% identical, and most preferably 95% or more identical to SEQ ID NO:2 or its orthologs.) Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zsig87. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other FASTA program parameters set as default.

The present invention includes nucleic acid molecules that encode a polypeptide having one or more conservative amino acid changes, compared with the amino acid sequence of SEQ ID NO:2. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. As used herein, the language “conservative amino acid substitution” refers to a substitution represented by a BLOSUM62 value of greater than −1. For example, an amino acid substitution is conservative if the substitution is characterized by a BLOSUM62 value of 0, 1, 2, or 3. Preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Variant zsig87 polypeptides or substantially homologous zsig87 polypeptides are characterized as having one or more amino acid substitutions, deletions or additions. These changes are preferably of a minor nature, that is conservative amino acid substitutions (see Table 4) and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides of from about 35 to about 90 amino acid residues that comprise a sequence that is at least 80%, preferably at least 90%, and more preferably 95% or more identical to the corresponding region of SEQ ID NO:2. Polypeptides comprising affinity tags can further comprise a proteolytic cleavage site between the zsig87 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

The present invention further provides a variety of other polypeptide fusions and related multimeric proteins comprising one or more polypeptide fusions. For example, a zsig87 polypeptide can be prepared as a fusion to a dimerizing protein as disclosed in U.S. Pat. Nos. 5,155,027 and 5,567,584. Preferred dimerizing proteins in this regard include immunoglobulin constant region domains. Immunoglobulin-zsig87 polypeptide fusions can be expressed in genetically engineered cells to produce a variety of multimeric zsig87 analogs. Auxiliary domains can be fused to zsig87 polypeptides to target them to specific cells, tissues, or macromolecules (e.g., collagen). For example, a zsig87 polypeptide or protein could be targeted to a predetermined cell type by fusing a zsig87 polypeptide to a ligand that specifically binds to a receptor on the surface of the target cell. In this way, polypeptides and proteins can be targeted for therapeutic or diagnostic purposes. A zsig87 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for zsig87 amino acid residues.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699-708, 1996. Sites of ligand-receptor or other biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related polypeptides.

Determination of amino acid residues that are within regions or domains that are critical to maintaining structural integrity can be determined. Within these regions one can determine specific residues that will be more or less tolerant of change and maintain the overall tertiary structure of the molecule. Methods for analyzing sequence structure include, but are not limited to, alignment of multiple sequences with high amino acid or nucleotide identity and computer analysis using available software (e.g., the Insight II® viewer and homology modeling tools; MSI, San Diego, Calif.), secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in zsig87 polypeptides so as to minimize disruption of higher order structure essential to biological activity. For example, when the zsig87 polypeptide comprises one or more helices, changes in amino acid residues will be made so as not to disrupt the helix geometry and other components of the molecule where changes in conformation abate some critical function, for example, binding of the molecule to its binding partners. The effects of amino acid sequence changes can be predicted by, for example, computer modeling as disclosed above or determined by analysis of crystal structure (see, e.g., Lapthom et al., Nat. Struct. Biol. 2:266-268, 1995). Other techniques that are well known in the art compare folding of a variant protein to a standard molecule (e.g., the native protein). For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same disulfide bonding pattern as the standard molecule folding would be affected. Another well known and accepted method for measuring folding is circular dichrosism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structural similarities between proteins and polypeptides (Schaanan et al., Science 257:961-964, 1992).

A Hopp/Woods hydrophilicity profile of the zsig87 protein sequence as shown in SEQ ID NO:2 can be generated (Hopp et al., Proc. Natl. Acad. Sci.78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored. Moreover, zsig87, hydrophilic regions include those described herein.

Those skilled in the art will recognize that hydrophilicity or hydrophobicity will be taken into account when designing modifications in the amino acid sequence of a zsig87 polypeptide, so as not to disrupt the overall structural and biological profile. Of particular interest for replacement are hydrophobic residues selected from the group consisting of Val, Leu and Ile or the group consisting of Met, Gly, Ser, Ala, Tyr and Trp. For example, hydrophobic residues tolerant of substitution could include such residues as shown in SEQ ID NO: 2. Cysteine residues, such as conserved cysteines at positions 28, 35, 39, 49, 55, and 56 of SEQ ID NO:2, are relatively intolerant of substitution.

The identities of essential amino acids can also be inferred from analysis of sequence similarity between other proteins with zsig87. Using methods such as “FASTA” analysis described previously, regions of high similarity are identified within a family of proteins and used to analyze amino acid sequence for conserved regions. An alternative approach to identifying a variant zsig87 polynucleotide on the basis of structure is to determine whether a nucleic acid molecule encoding a potential variant zsig87 polynucleotide can hybridize to a nucleic acid molecule having the nucleotide sequence of SEQ ID NO:1, as discussed above.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699 (1996).

The present invention also includes functional fragments of zsig87 polypeptides and nucleic acid molecules encoding such functional fragments. A “functional” zsig87 or fragment thereof defined herein is characterized by its proliferative or differentiating activity, by its ability to induce or inhibit specialized cell functions, or by its ability to bind specifically to an anti-zsig87 antibody or zsig87 receptor (either soluble or immobilized). The present invention further provides fusion proteins encompassing: (a) polypeptide molecules comprising one or more of the motifs described above; and (b) functional fragments comprising one or more of these motifs. The other polypeptide portion of the fusion protein may be contributed by a related, non-native and/or an unrelated secretory signal peptide that facilitates secretion of the fusion protein.

Routine deletion analyses of nucleic acid molecules can be performed to obtain functional fragments of a nucleic acid molecule that encodes a zsig87 polypeptide. As an illustration, DNA molecules having the nucleotide sequence of SEQ ID NO:1 or fragments thereof, can be digested with Bal31 nuclease to obtain a series of nested deletions. These DNA fragments are then inserted into expression vectors in proper reading frame, and the expressed polypeptides are isolated and tested for zsig87 activity, or for the ability to bind anti-zsig87 antibodies or zsig87 receptor. One alternative to exonuclease digestion is to use oligonucleotide-directed mutagenesis to introduce deletions or stop codons to specify production of a desired zsig87 fragment. Alternatively, particular fragments of a zsig87 polynucleotide can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993); Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987); Herschman, “The EGF Receptor,” in Control of Animal Cell Proliferation 1, Boynton et al., (eds.) pages 169-199 (Academic Press 1985); Coumailleau et al., J. Biol. Chem. 270:29270 (1995); Fukunaga et al., J. Biol. Chem. 270:25291 (1995); Yamaguchi et al., Biochem. Pharmacol. 50:1295 (1995); and Meisel et al., Plant Molec. Biol. 30:1 (1996).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed zsig87 DNA and polypeptide sequences can be generated through DNA shuffling as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode active polypeptides (e.g., secreted and detected by antibodies; or measured by a signal transduction type assay) can be recovered from the host cells and rapidly sequenced using modern equipment. These methods allow the rapid determination of the importance of individual amino acid residues in a polypeptide of interest, and can be applied to polypeptides of unknown structure.

In addition, the proteins of the present invention (or polypeptide fragments thereof) can be joined to other bioactive molecules, particularly other cytokines, to provide multi-functional molecules. For example, one or more domains, hydrophilic regions, or regions containing motifs 1-4 from zsig87 can be joined to other proteins to enhance their biological properties or efficiency of production.

The present invention thus provides a series of novel, hybrid molecules in which a segment comprising one or more of the domains, hydrophilic regions, or regions containing motifs 1-4 of zsig87 is fused to another polypeptide. Fusion is preferably done by splicing at the DNA level to allow expression of chimeric molecules in recombinant production systems. The resultant molecules are then assayed for such properties as improved solubility, improved stability, prolonged clearance half-life, improved expression and secretion levels, and pharmacodynamics. Such hybrid molecules may further comprise additional amino acid residues (e.g. a polypeptide linker) between the component proteins or polypeptides.

Using the methods discussed herein, one of ordinary skill in the art can identify and/or prepare a variety of polypeptides that are substantially similar to SEQ ID NO:2 or allelic variants thereof and retain the properties of the wild-type protein. For example, using the methods described above, one could identify a receptor binding domain on zsig87; an extracellular ligand-binding domain of a receptor for zsig87; heterodimeric and homodimeric binding domains; other functional or structural domains; affinity tags; or other domains important for protein-protein interactions or signal transduction. Such polypeptides may also include additional polypeptide segments as generally disclosed above.

For any zsig87 polypeptide, including variants and fusion proteins, one of ordinary skill in the art can readily generate a fully degenerate polynucleotide sequence encoding that variant using the information set forth in Tables 1 and 2 above.

The zsig87 polypeptides of the present invention, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a zsig87 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zsig87 polypeptide into the secretory pathway of a host cell, a secretory signal sequence (also known as a leader sequence, prepro sequence or pre sequence) is provided in the expression vector. The secretory signal sequence may be that of zsig87, or may be derived from another secreted protein (e.g., t-PA) or synthesized de novo. The secretory signal sequence is operably linked to the zsig87 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

Alternatively, the secretory signal sequence contained in the polypeptides of the present invention is used to direct other polypeptides into the secretory pathway. The present invention provides for such fusion polypeptides. A signal fusion polypeptide can be made wherein a secretory signal sequence derived from residue 1 (Met) to residue 15 (Glu) of SEQ ID NO:2 is be operably linked to another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein. Such fusions may be used in vivo or in vitro to direct peptides through the secretory pathway.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured, for example in the presence of the selective agent, and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used. In addition to selection of transfectants by agents, alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols, Methods in Molecular Biology, Totowa, N.J., Humana Press, 1995. The second method of making recombinant baculovirus utilizes a transposon-based system described by Luckow (Luckow, V. A, et al., J Virol 67:4566-79, 1993). This system is sold in the Bac-to-Bac kit (Life Technologies, Rockville, Md.). This system utilizes a transfer vector, pFastBac1? (Life Technologies) containing a Tn7 transposon to move the DNA encoding the zsig87 polypeptide into a baculovirus genome maintained in E. coli as a large plasmid called a “bacmid.” The pFastBacl? transfer vector utilizes the AcNPV polyhedrin promoter to drive the expression of the gene of interest, in this case zsig87. However, pFastBacl? can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol. Chem. 270:1543-9, 1995. In such transfer vector constructs, a short or long version of the basic protein promoter can be used. Moreover, transfer vectors can be constructed which replace the native zsig87 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native zsig87 secretory signal sequence. In addition, transfer vectors can include an in-frame fusion with DNA encoding an epitope tag at the C- or N-terminus of the expressed zsig87 polypeptide, for example, a Glu-Glu epitope tag (Grussenmeyer, T. et al., Proc. Natl. Acad. Sci. 82:7952-4, 1985). Using a technique known in the art, a transfer vector containing zsig87 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zsig87 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO? cell line (Invitrogen) derived from Trichoplusia ni (U.S. Pat. No. 5,300,435). Commercially available serum-free media are used to grow and maintain the cells. Suitable media are Sf900 II? (Life Technologies) or ESF 921? (Expression Systems) for the Sf9 cells; and Ex-celO405? (JRH Biosciences, Lenexa, Kans.) or Express FiveO? (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D.R. et al., ibid.; Richardson, C. D., ibid.). Subsequent purification of the zsig87 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillennondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a zsig87 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto? Peptone (Difco Laboratories, Detroit, Mich.), 1% Bacto? yeast extract (Difco Laboratories), 0.004% adenine and 0.006% L-leucine).

It is preferred to purify the polypeptides of the present invention to ≧80% purity, more preferably to ≧90% purity, even more preferably ≧95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant zsig87 polypeptides (or chimeric zsig87 polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their biological and structural properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Within additional embodiments of the invention, a fusion of the polypeptide of interest and an affinity tag (e.g., maltose-binding protein, an immunoglobulin domain) may be constructed to facilitate purification.

Moreover, using methods described in the art, polypeptide fusions, or hybrid zsig87 proteins, are constructed using regions or domains of zsig87 in combination with those of paralogs, orthologs, or heterologous proteins (Sambrook et al., ibid., Altschul et al., ibid., Picard. D., Cur. Opin. Biology, 5:511-515, 1994, and references therein). These methods allow the determination of the biological importance of larger domains or regions in a polypeptide of interest. Such hybrids may alter reaction kinetics, binding, constrict or expand the substrate specificity, or alter tissue and cellular localization of a polypeptide, and can be applied to polypeptides of unknown structure.

Fusion polypeptides can be prepared by methods known to those skilled in the art by preparing each component of the fusion protein and chemically conjugating them. Alternatively, a polynucleotide encoding one or more components of the fusion protein in the proper reading frame can be generated using known techniques and expressed by the methods described herein. For example, part or all of a domain(s) conferring a biological function may be swapped between zsig87 of the present invention with the functionally equivalent domain(s) from another protein. Such domains include, but are not limited to, the secretory signal sequence, and motifs 1 through 4. Such fusion proteins would be expected to have a biological functional profile that is the same or similar to polypeptides of the present invention or other similar proteins (e.g. paralogs, or orthologs) or heterologous proteins, depending on the fusion constructed. Moreover, such fusion proteins may exhibit other properties as disclosed herein.

Standard molecular biological and cloning techniques can be used to swap the equivalent domains between the zsig87 polypeptide and those polypeptides to which they are fused. Generally, a DNA segment that encodes a domain of interest, e.g., a zsig87 domain described herein, is operably linked in frame to at least one other DNA segment encoding an additional polypeptide (for instance an analogous domain or region in a similar protein), and inserted into an appropriate expression vector, as described herein. Generally DNA constructs are made such that the several DNA segments that encode the corresponding regions of a polypeptide are operably linked in frame to make a single construct that encodes the entire fusion protein, or a functional portion thereof. For example, a DNA construct would encode from N-terminus to C-terminus a fusion protein comprising a signal polypeptide followed by a mature polypeptide; or a DNA construct would encode from N-terminus to C-terminus a fusion protein comprising a signal polypeptide followed by a mature protein. Such fusion proteins can be expressed, isolated, and assayed for activity as described herein. Moreover, such fusion proteins can be used to express and secrete fragments of the zsig87 polypeptide, to be used, for example to inoculate an animal to generate anti-zsig87 antibodies as described herein. For example a secretory signal sequence can be operably linked to the N-terminal domain, conserved domain, mature zsig87 polypeptide, C-terminal domain, or a combination thereof (e.g., operably linked polypeptides comprising the N-terminal domain fused to the conserved domain, or zsig87 polypeptide fragments described herein), to secrete a fragment of zsig87 polypeptide that can be purified as described herein and serve as an antigen to be inoculated into an animal to produce anti-zsig87 antibodies, as described herein.

Zsig87 polypeptides or fragments thereof may also be prepared through chemical synthesis. zsig87 polypeptides may be monomers or multimers; glycosylated or non-glycosylated; pegylated or non-pegylated; and may or may not include an initial methionine amino acid residue.

Polypeptides of the present invention can also be synthesized by exclusive solid phase synthesis, partial solid phase methods, fragment condensation or classical solution synthesis. Methods for synthesizing polypeptides are well known in the art. See, for example, Merrifield, J. Am. Chem. Soc. 85:2149, 1963; Kaiser et al., Anal. Biochem. 34:595, 1970. After the entire synthesis of the desired peptide on a solid support, the peptide-resin is with a reagent which cleaves the polypeptide from the resin and removes most of the side-chain protecting groups. Such methods are well established in the art.

The activity of molecules of the present invention can be measured using a variety of assays that measure for example, signal transduction, cell motility, steroidogenesis, mitogenesis or binding. Such assays are well known in the art.

The zsig87 polypeptides of the present invention can be used to study pancreatic cell proliferation or differentiation. Such methods of the present invention generally comprise incubating α cells, β cells, δ cells, F cells and acinar cells in the presence and absence of zsig87 polypeptide, monoclonal antibody, agonist or antagonist thereof and observing changes in islet cell proliferation or differentiation.

A further aspect of the invention provides a method for studying insulin. Such methods of the present invention comprise incubating adipocytes in a culture medium comprising zsig87 polypeptide, monoclonal antibody, agonist or antagonist thereof ? insulin and observing changes in adipocyte protein secretion or differentiation.

The present invention also provides methods of studying mammalian cellular metabolism. Such methods of the present invention comprise incubating cells to be studied, for example, an appropriate human cell line, zsig87 polypeptide, monoclonal antibody, agonist or antagonist thereof, and observing changes in adipogenesis, gluconeogenesis, glycogenolysis, lipogenesis, glucose uptake, or the like.

Also, zsig87 polypeptides, agonists or antagonists thereof may be therapeutically useful for promoting wound healing, for example, in the pancreas. To verify the presence of this capability in zsig87 polypeptides, agonists or antagonists of the present invention, such zsig87 polypeptides, agonists or antagonists are evaluated with respect to their ability to facilitate wound healing according to procedures known in the art. If desired, zsig87 polypeptide performance in this regard can be compared to growth factors, such as EGF, NGF, TGF-α, TGF-β, insulin, IGF-I, IGF-II, fibroblast growth factor (FGF) and the like. In addition, zsig87 polypeptides or agonists or antagonists thereof may be evaluated in combination with one or more growth factors to identify synergistic effects.

In addition, zsig87 polypeptides, agonists or antagonists thereof may be therapeutically useful for anti-microbial applications. To verify the presence of this capability in zsig87 polypeptides, agonists or antagonists of the present invention, such zsig87 polypeptides, agonists or antagonists are evaluated with respect to their antimicrobial properties according to procedures known in the art. See, for example, Barsum et al., Eur. Respir. J. 8(5): 709-14, 1995; Sandovsky-Losica et al., J. Med. Vet. Mycol (England) 28(4): 279-87, 1990; Mehentee et al., J. Gen. Microbiol (England) 135 (Pt. 8): 2181-8, 1989; Segal and Savage, Journal of Medical and Veterinary Mycology 24: 477-479, 1986 and the like. If desired, zsig87 polypeptide performance in this regard can be compared to proteins known to be functional in this regard, such as proline-rich proteins, lysozyme, histatins, lactoperoxidase or the like. In addition, zsig87 polypeptides or agonists or antagonists thereof may be evaluated in combination with one or more antimicrobial agents to identify synergistic effects.

Anti-microbial protective agents may be directly acting or indirectly acting. Such agents operating via membrane association or pore forming mechanisms of action directly attach to the offending microbe. Anti-microbial agents can also act via an enzymatic mechanism, breaking down microbial protective substances or the cell wall/membrane thereof. Anti-microbial agents, capable of inhibiting microorganism proliferation or action or of disrupting microorganism integrity by either mechanism set forth above, are useful in methods for preventing contamination in cell culture by microbes susceptible to that anti-microbial activity. Such techniques involve culturing cells in the presence of an effective amount of said zsig87 polypeptide, or an agonist or antagonist thereof.

Also, zsig87 polypeptides or agonists thereof may be used as cell culture reagents in in vitro studies of exogenous microorganism infection, such as bacterial, viral or fungal infection. Such moieties may also be used in in vivo animal models of infection. Also, the microorganism-adherence properties of zsig87 polypeptides or agonists thereof can be studied under a variety of conditions in binding assays and the like.

Proteins of the present invention are useful for example, in treating ovarian, testicular, pancreatic, ocular, immune, lymphatic or blood or bone disorders, can be measured in vitro using cultured cells or in vivo by administering molecules of the claimed invention to the appropriate animal model. For instance, host cells expressing a secreted form of zsig87 polypeptide may be embedded in an alginate environment and injected (implanted) into recipient animals. Alginate-poly-L-lysine microencapsulation, permselective membrane encapsulation and diffusion chambers are a means to entrap transfected mammalian cells or primary mammalian cells to permit the diffusion of proteins and other macromolecules secreted or released by the captured cells to the recipient animal. Most importantly, the capsules mask and shield the foreign, embedded cells from the recipient animal's immune response. Such encapsulations can extend the life of the injected cells from a few hours or days (naked cells) to several weeks (embedded cells). Alginate threads provide a simple and quick means for generating embedded cells and testing, in vivo, the proteins secreted therefrom. The materials needed to generate the alginate threads are known in the art. In an exemplary procedure, 3% alginate is prepared in sterile H₂O, and sterile filtered. Just prior to preparation of alginate threads, the alginate solution is again filtered. An approximately 50% cell suspension (containing about 5×10⁵ to about 5×10⁷ cells/ml) is mixed with the 3% alginate solution. One ml of the alginate/cell suspension is extruded into a 100 mM sterile filtered CaCl₂ solution over a time period of ˜15 min, forming a “thread”. The extruded thread is then transferred into a solution of 50 mM CaCl₂, and then into a solution of 25 mM CaCl₂. The thread is then rinsed with deionized water before coating the thread by incubating in a 0.01% solution of poly-L-lysine. Finally, the thread is rinsed with Lactated Ringer's Solution and drawn from solution into a syringe barrel (without needle). A large bore needle is then attached to the syringe, and the thread is intraperitoneally injected into a recipient in a minimal volume of the Lactated Ringer's Solution.

An in vivo approach for assaying proteins of the present invention involves viral delivery systems. Exemplary viruses for this purpose include adenovirus, herpesvirus, retroviruses, vaccinia virus, and adeno-associated virus (AAV). Adenovirus, a double-stranded DNA virus, is currently the best studied gene transfer vector for delivery of heterologous nucleic acid (for review, see T. C. Becker et al., Meth. Cell Biol. 43:161-89, 1994; and J. T. Douglas and D. T. Curiel, Science & Medicine 4:44-53, 1997). The adenovirus system offers several advantages: (i) adenovirus can accommodate relatively large DNA inserts; (ii) can be grown to high-titer; (iii) infect a broad range of mammalian cell types; and (iv) can be used with many different promoters including ubiquitous, tissue specific, and regulatable promoters. Also, because adenoviruses are stable in the bloodstream, they can be administered by intravenous injection.

Using adenovirus vectors where portions of the adenovirus genome are deleted, inserts are incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293 cells can be grown as adherent cells or in suspension culture at relatively high cell density to produce significant amounts of protein (See Gamier et al., Cytotechnol. 15:145-55, 1994). With either protocol, an expressed, secreted heterologous protein can be repeatedly isolated from the cell culture supernatant, lysate, or membrane fractions depending on the disposition of the expressed protein in the cell. Within the infected 293 cell production protocol, non-secreted proteins may also be effectively obtained.

In view of the tissue distribution observed for zsig87, agonists (including the natural ligand/substrate/cofactor/etc.) and antagonists have enormous potential in both in vitro and in vivo applications. Compounds identified as zsig87 agonists are useful for stimulating cell growth or signal transduction in vitro and in vivo. For example, zsig87zsig87 and agonist compounds are useful as components of defined cell culture media, and may be used alone or in combination with other cytokines and hormones to replace serum that is commonly used in cell culture. Agonists are thus useful in specifically promoting the growth and/or development of cells in culture. Considering the high expression of zsig87 ovary and pancreas, zsig87 polypeptides and zsig87 agonists may be particularly useful as research reagents, particularly for the growth of pancreatic cell types and ovarian cell lines, human eggs, cells from animal embryos or primary cultures derived from these tissues, T-cells, B-cells, and other cells of the lymphoid and myeloid lineages and hematopoetic lineages. As such, zsig87 polypeptide can be provided as a supplement in cell culture medium. As such, zsig87 polypeptide can be provided as a supplement in cell culture medium.zsig87.

Antagonists are also useful as research reagents for characterizing sites of ligand-receptor interaction. Inhibitors of zsig87 activity (zsig87 antagonists) include anti-zsig87 antibodies and soluble proteins which bind zsig87 polypeptide. Inhibitors of zsig87 activity (zsig87 antagonists) include anti-zsig87 antibodies and soluble zsig87 receptors, as well as other peptidic and non-peptidic agents (including ribozymes).

Zsig87 polypeptide can be used to identify inhibitors (antagonists) of its activity. Test compounds are added to the biological or biochemical assays disclosed herein to identify compounds that inhibit the activity of zsig87. In addition to those assays disclosed herein, samples can be tested for inhibition of zsig87 activity within a variety of assays designed to measure receptor binding or the stimulation/inhibition of zsig87-dependent cellular responses. For example, zsig87-responsive cell lines can be transfected with a reporter gene construct that is responsive to a zsig87-stimulated cellular pathway. Reporter gene constructs of this type are known in the art, and will generally comprise a zsig87-DNA response element operably linked to a gene encoding an assay detectable protein, such as luciferase. DNA response elements can include, but are not limited to, cyclic AMP response elements (CRE), hormone response elements (HRE) insulin response element (IRE) (Nasrin et al., Proc. Natl. Acad. Sci. USA 87:5273-7, 1990) and serum response elements (SRE) (Shaw et al., Cell 56: 563-72, 1989). Cyclic AMP response elements are reviewed in Roestler et al., J. Biol. Chem. 263 (19):9063-6; 1988 and Habener, Molec. Endocrinol. 4 (8):1087-94; 1990. Hormone response elements are reviewed in Beato, Cell 56:335-44; 1989. Candidate compounds that serve as test samples including solutions, mixtures or extracts, are tested for the level of response to the zsig87 polypeptide. The ability of a test sample to inhibit the activity of zsig87 polypeptide on the target cells as evidenced by a decrease in zsig87 stimulation of reporter gene expression in the presence of a test sample relative to a control which was cultured in the absence of a test sample. Assays of this type will detect compounds that directly block zsig87 binding to cell-surface receptors, e.g., dimerization, as well as compounds that block processes in the cellular pathway subsequent to receptor-ligand binding. Alternatively, compounds or other samples can be tested for direct blocking of zsig87 binding to receptor using zsig87 tagged with a detectable label (e.g., ¹²⁵I, biotin, horseradish peroxidase, FITC, and the like). Within assays of this type, the ability of a test sample to inhibit the binding of labeled zsig87 to the receptor is indicative of inhibitory activity, which can be confirmed through secondary assays. Receptors used within binding assays may be cellular receptors or isolated, immobilized receptors.

Alternatively, the above methodology may be used to identify agonists of zsig87 activity. Candidate compounds serving as test samples including solutions, mixtures or extracts, are tested for the ability to mimic the activity of zsig87 polypeptide on the target cells as evidenced by stimulation of reporter gene expression in the presence of a test sample and the absence of zsig87, relative to a control (cultured in the absence of a test sample and the absence of zsig87 polypeptide), using assays as described above.

A zsig87 polypeptide can be expressed as a fusion with an immunoglobulin heavy chain constant region, typically an F_{c} fragment, which contains two constant region domains and lacks the variable region. Methods for preparing such fusions are disclosed in U.S. Pat. Nos. 5,155,027 and 5,567,584. Such fusions are typically secreted as multimeric molecules wherein the Fc portions are disulfide bonded to each other and two non-Ig polypeptides are arrayed in closed proximity to each other. Fusions of this type can be used to affinity purify ligand, as an in vitro assay tool, or an antagonist of zsig87. For use in assays, the chimeras are bound to a support via the F_{c} region and used in an ELISA format.

A zsig87 polypeptide can also be used for purification of receptor or polypeptides to which it binds. The zsig87 polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and membrane fractions containing receptors are passed through the column one or more times to allow the receptor to bind to the ligand zsig87 polypeptide. The receptor is then eluted using changes in salt concentration, chaotropic agents (guanidine HCl), or pH to disrupt ligand-receptor binding.

An assay system that uses a ligand-binding receptor (or an antibody, one member of a complement/anti-complement pair) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

Ligand-binding receptor polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

As a ligand, the activity of zsig87 polypeptide can be measured by a silicon-based biosensor microphysiometer which measures the extracellular acidification rate or proton excretion associated with receptor binding and subsequent physiologic cellular responses. An exemplary device is the Cytosensor? Microphysiometer manufactured by Molecular Devices, Sunnyvale, Calif. A variety of cellular responses, such as cell proliferation, ion transport, energy production, inflammatory response, regulatory and receptor activation, and the like, can be measured by this method. See, for example, McConnell, H. M. et al., Science 257:1906-1912, 1992; Pitchford, S. et al., Meth. Enzymol. 228:84-108, 1997; Arimilli, S. et al., J. Immunol. Meth. 212:49-59, 1998; Van Liefde, I. et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including zsig87 polypeptide, its agonists, or antagonists. Preferably, the microphysiometer is used to measure responses of a zsig87-responsive eukaryotic cell, compared to a control eukaryotic cell that does not respond to zsig87 polypeptide. Zsig87-responsive eukaryotic cells comprise cells into which a receptor for zsig87 has been transfected creating a cell that is responsive to zsig87; or cells naturally responsive to zsig87 such as cells derived from testis tissue. Differences, measured by a change, for example, an increase or diminution in extracellular acidification, in the response of cells exposed to zsig87 polypeptide, relative to a control not exposed to zsig87, are a direct measurement of zsig87-modulated cellular responses. Moreover, such zsig87-modulated responses can be assayed under a variety of stimuli. Using the microphysiometer, there is provided a method of identifying agonists of zsig87 polypeptide, comprising providing cells responsive to a zsig87 polypeptide, culturing a first portion of the cells in the absence of a test compound, culturing a second portion of the cells in the presence of a test compound, and detecting a change, for example, an increase or diminution, in a cellular response of the second portion of the cells as compared to the first portion of the cells. The change in cellular response is shown as a measurable change extracellular acidification rate. Moreover, culturing a third portion of the cells in the presence of zsig87 polypeptide and the absence of a test compound can be used as a positive control for the zsig87-responsive cells, and as a control to compare the agonist activity of a test compound with that of the zsig87 polypeptide. Moreover, using the microphysiometer, there is provided a method of identifying antagonists of zsig87 polypeptide, comprising providing cells responsive to a zsig87 polypeptide, culturing a first portion of the cells in the presence of zsig87 and the absence of a test compound, culturing a second portion of the cells in the presence of zsig87 and the presence of a test compound, and detecting a change, for example, an increase or a diminution in a cellular response of the second portion of the cells as compared to the first portion of the cells. The change in cellular response is shown as a measurable change extracellular acidification rate. Antagonists and agonists, for zsig87 polypeptide, can be rapidly identified using this method.

Moreover, zsig87 can be used to identify cells, tissues, or cell lines which respond to a zsig87-stimulated pathway. The microphysiometer, described above, can be used to rapidly identify ligand-responsive cells, such as cells responsive to zsig87 of the present invention. Cells can be cultured in the presence or absence of zsig87 polypeptide. Those cells which elicit a measurable change in extracellular acidification in the presence of zsig87 are responsive to zsig87. Such cell lines, can be used to identify antagonists and agonists of zsig87 polypeptide as described above.

At least one tissue in which zsig87 is expressed is a tissue that contracts. For example a contractile tissue in which zsig87 is expressed includes tissues in testis, e.g., vas deferens. Other contractile tissues include prostate tissues; gastrointestinal tissues, e.g., colon and small intestine; and heart. The effects of zsig87 polypeptide, its antagonists and agonists, on tissue contractility can be measured in vitro using a tensiometer with or without electrical field stimulation. Such assays are known in the art and can be applied to tissue samples, such as aortic rings, vas deferens, ilium, uterine and other contractile tissue samples, as well as to organ systems, such as atria, and can be used to determine whether zsig87 polypeptide, its agonists or antagonists, enhance or depress contractility. Molecules of the present invention are hence useful for treating dysfunction associated with contractile tissues or can be used to suppress or enhance contractility in vivo. As such, molecules of the present invention have utility in treating cardiovascular disease, infertility, in vitro fertilization, birth control, treating impotence or other male reproductive dysfunction, as well as inducing birth.

The effect of the zsig87 polypeptides, antagonists and agonists of the present invention on contractility of tissues including uterus, prostate, testis, gastrointestinal tissues, and heart can be measured in a tensiometer that measures contractility and relaxation in tissues. See, Dainty et al., J. Pharmacol. 100:767, 1990; Rhee et al., Neurotox. 16: 179, 1995; Anderson, M. B., Endocrinol. 114:364-368, 1984; and Downing, S. J. and Sherwood, O. D, Endocrinol. 116:1206-1214, 1985. For example, measuring vasodilatation of aortic rings is well known in the art. Briefly, aortic rings are taken from 4 month old Sprague Dawley rats and placed in a buffer solution, such as modified Krebs solution (118.5 mM NaCl, 4.6 mM KCl, 1.2 mM MgSO₄.7H₂O, 1.2 mM KH₂PO₄, 2.5 mM CaCl₂.2H₂O, 24.8 mM NaHCO₃ and 10 mM glucose). One of skill in the art would recognize that this method can be used with other animals, such as rabbits, other rat strains, Guinea pigs, and the like. The rings are then attached to an isometric force transducer (Radnoti Inc., Monrovia, Calif.) and the data recorded with a Ponemah physiology platform (Gould Instrument systems, Inc., Valley View, Ohio) and placed in an oxygenated (95% O₂, 5% CO₂) tissue bath containing the buffer solution. The tissues are adjusted to 1 gram resting tension and allowed to stabilize for about one hour before testing. The integrity of the rings can be tested with norepinepherin (Sigma Co., St. Louis, Mo.) and Carbachol, a muscarinic acetylcholine agonist (Sigma Co.). After integrity is checked, the rings are washed three times with fresh buffer and allowed to rest for about one hour. To test a sample for vasodilatation, or relaxation of the aortic ring tissue, the rings are contracted to two grams tension and allowed to stabilize for fifteen minutes. A zsig87 polypeptide sample is then added to 1, 2 or 3 of the 4 baths, without flushing, and tension on the rings recorded and compared to the control rings containing buffer only. Enhancement or relaxation of contractility by zsig87 polypeptides, their agonists and antagonists is directly measured by this method, and it can be applied to other contractile tissues such as uterus, prostate, and testis.

The activity of molecules of the present invention can be measured using a variety of assays that measure stimulation of gastrointestinal cell contractility, modulation of nutrient uptake and/or secretion of digestive enzymes. Of particular interest are changes in contractility of smooth muscle cells. For example, the contractile response of segments of mammalian duodenum or other gastrointestinal smooth muscles tissue (Depoortere et al., J. Gastrointestinal Motility 1:150-159, 1989, incorporated herein by reference). An exemplary in vivo assay uses an ultrasonic micrometer to measure the dimensional changes radially between commissures and longiturdinally to the plane of the valve base (Hansen et al., Society of Thoracic Surgeons 60:S384-390, 1995).

Gastric motility is generally measured in the clinical setting as the time required for gastric emptying and subsequent transit time through the gastrointestinal tract. Gastric emptying scans are well known to those skilled in the art, and briefly, comprise use of an oral contrast agent, such as barium, or a radiolabeled meal. Solids and liquids can be measured independently. A test food or liquid is radiolabeled with an isotope (e.g. ^{99m}Tc), and after ingestion or administration, transit time through the gastrointestinal tract and gastric emptying are measured by visualization using gamma cameras (Meyer et al., Am. J. Dig. Dis. 21:296, 1976; Collins et al., Gut 24:1117, 1983; Maughan et al., Diabet. Med. 13 9 Supp. 5:S6-10, 1996 and Horowitz et al., Arch. Intern. Med. 145:1467-1472, 1985). These studies may be performed before and after the administration of a promotility agent to quantify the efficacy of the drug.

The tissue specificity of zsig87 expression suggests a role in spermatogenesis, a process that is remarkably similar to the development of blood cells (hematopoiesis). Briefly, spermatogonia undergo a maturation process similar to the differentiation of hematopoietic stem cells. In view of the tissue specificity observed for zsig87, agonists and antagonists have enormous potential in both in vitro and in vivo applications. Zsig87 polypeptides, agonists and antagonists may also prove useful in modulating spermatogenesis and thus aid in overcoming infertility. Antagonists are useful as research reagents for characterizing sites of ligand-receptor interaction. In vivo, zsig87 polypeptides, agonists or antagonists may find application in the treatment of male infertility or as a male contraceptive agents.

The zsig87 polypeptides, antagonists of agonists, of the present invention can also modulate sperm capacitation. Before reaching the oocyte or egg and initiating an egg-sperm interaction, the sperm must be activated. The sperm undergo a gradual capacitation, lasting up to 3 or 4 hours in vitro, during which the plasma membrane of the sperm head and the outer acrosomal membrane fuse to form vesicles that facilitate the release of acrosomal enzymes. The acrosomal membrane surrounds the acrosome or acrosomal cap which is located at the anterior end of the nucleus in the sperm head. In order for the sperm to fertilize egg the sperm must penetrate the oocyte. To enable this process the sperm must undergo acrosomal exocytosis, also known as the acrosomal reaction, and release the acrosomal enzymes in the vicinity of the oocyte. These enzymes enable the sperm to penetrate the various oocyte layers, (the cumulus oophorus, the corona radiata and the zona pellucida). The released acrosomal enzymes include hyaluronidase and proacrosin, in addition to other enzymes such as proteases. During the acrosomal reaction, proacrosin is converted to acrosin, the active form of the enzyme, which is required for and must occur before binding and penetration of the zona pellucida is possible. A combination of the acrosomal lytic enzymes and sperm tail movements allow the sperm to penetrate the oocyte layers. Numerous sperm must reach the egg and release acrosomal enzymes before the egg can finally be fertilized. Only one sperm will successfully bind to, penetrate and fertilize the egg, after which the zona hardens so that no other sperm can penetrate the egg (Zaneveld, in Male Infertility Chapter 11, Comhaire (Ed.), Chapman & Hall, London, 1996). Peptide hormones, such as insulin homologs are associated with sperm activation and egg-sperm interaction. For instance, capacitated sperm incubated with relaxin show an increased percentage of progressively motile sperm, increased zona penetration rates, and increased percentage of viable acrosome-reacted sperm (Carrell et al., Endocr. Res. 21:697-707, 1995). Similarity of the zsig87 polypeptide structure with peptide hormones and localization of Zsig87 to the testis, prostate and uterus suggests that the zsig87 polypeptides described herein play a role in these and other reproductive processes.

Accordingly, proteins of the present invention can have applications in enhancing fertilization during assisted reproduction in humans and in animals. Such assisted reproduction methods are known in the art and include artificial insemination, in vitro fertilization, embryo transfer and gamete intrafallopian transfer. Such methods are useful for assisting men and women who have physiological or metabolic disorders preventing natural conception or can be used to enhance in vitro fertilization. Such methods are also used in animal breeding programs, such as for livestock breeding and could be used as methods for the creation of transgenic animals. Proteins of the present invention can be combined with sperm, an egg or an egg-sperm mixture prior to fertilization of the egg. In some species, sperm capacitate spontaneously during in vitro fertilization procedures, but normally sperm capacitate over an extended period of time both in vivo and in vitro. It is advantageous to increase sperm activation during such procedures to enhance the likelihood of successful fertilization. The washed sperm or sperm removed from the seminal plasma used in such assisted reproduction methods has been shown to have altered reproductive functions, in particular, reduced motility and zona interaction. To enhance fertilization during assisted reproduction methods sperm is capacitated using exogenously added compounds. Suspension of the sperm in seminal plasma from normal subjects or in a “capacitation media” containing a cocktail of compounds known to activate sperm, such as caffeine, dibutyl cyclic adenosine monophosphate (dbcAMP) or theophylline, have resulted in improved reproductive function of the sperm, in particular, sperm motility and zonae penetration (Park et al., Am. J. Obstet. Gynecol. 158:974-9, 1988; Vandevoort et al., Mol. Repro. Develop. 37:299-304, 1993; Vandevoort and Overstreet, J. Androl. 16:327-33, 1995). The presence of immunoreactive relaxin in vivo and in association with cryopreserved semen, was shown to significantly increase sperm motility (Juang et al., Anim. Reprod. Sci. 20:21-9, 1989; Juang et al., Anim. Reprod. Sci. 22:47-53, 1990). Porcine relaxin stimulated sperm motility in cryopreserved human sperm (Colon et al., Fertil. Steril. 46:1133-39, 1986; Lessing et al., Fertil. Steril. 44:406-9, 1985) and preserved ability of washed human sperm to penetrate cervical mucus in vitro (Brenner et al., Fertil. Steril. 42:92-6, 1984). Polypeptides of the present invention can used in such methods to enhance viability of cryopreserved sperm, enhance sperm motility and enhance fertilization, particularly in association with methods of assisted reproduction.

In cases where pregnancy is not desired, zsig87 polypeptide or polypeptide fragments may function as germ-cell-specific antigens for use as components in “immunocontraceptive” or “anti-fertility” vaccines to induce formation of antibodies and/or cell mediated immunity to selectively inhibit a process, or processes, critical to successful reproduction in humans and animals. The use of sperm and testis antigens in the development of immunocontraceptives have been described (O'Hern et al., Biol Reprod. 52:311-39, 1995; Diekman and Herr, Am. J. Reprod. Immunol. 37:111-17, 1997; Zhu and Naz, Proc. Natl. Acad. Sci. USA 94:4704-9,1997). A vaccine based on human chorionic gonadotrophin (HCG) linked to a diphtheria or tetanus carrier was in clinical trials (Talwar et al., Proc. Natl. Acad. Sci. USA 91:8532-36, 1994). A single injection resulted in production of high titer antibodies that persisted for nearly a year in rabbits (Stevens, Am. J. Reprod. Immunol. 29:176-88, 1993). Such methods of immunocontraception using vaccines would include a zsig87 testes-specific protein or fragment thereof. The Zsig87 protein or fragments can be conjugated to a carrier protein or peptide, such as tetanus or diphtheria toxoid. An adjuvant, as described above, can be included and the protein or fragment can be noncovalently associated with other molecules to enhance intrinsic immunoreactivity. Methods for administration and methods for determining the number of administrations are known in the art. Such a method might include a number of primary injections over several weeks followed by booster injections as needed to maintain a suitable antibody titer.

Regulation of reproductive function in males and females is controlled in part by feedback inhibition of the hypothalamus and anterior pituitary by blood-borne hormones. Testis proteins, such as activins and inhibins, have been shown to regulate secretion of active molecules including follicle stimulating hormone (FSH) from the pituitary (Ying, Endodcr. Rev. 9:267-93, 1988; Plant et al., Hum. Reprod. 8:41-44,1993). Inhibins, also expressed in the ovaries, have been shown to regulate ovarian functions (Woodruff et al., Endocr. 132:2332-42,1993; Russell et al., J. Reprod. Fertil. 100:115-22, 1994). Relaxin has been shown to be a systemic and local acting hormone regulating follicular and uterine growth (Bagnell et al., J. Reprod. Fertil. 48:127-38, 1993). As such, the polypeptides of the present invention may also have effects on female gametes and reproductive tract. These functions may also be associated with zsig87 polypeptides and may be used to regulate testicular or ovarian functions.

The polypeptides, antagonists, agonists, nucleic acid and/or antibodies of the present invention may be used in treatment of disorders associated with gonadal development, pregnancy, pubertal changes, menopause, ovarian cancer, fertility, ovarian function, polycystic ovarian syndrome, uterine cancer, endometriosis, libido, mylagia and neuralgia associated with reproductive phenomena, male sexual dysfunction, impotency, prostate cancer, testicular cancer, stomach cancer, gastrointestinal mobility and dysfunction. The molecules of the present invention may used to modulate or to treat or prevent development of pathological conditions in such diverse tissue as testis and the immune system. In particular, certain syndromes or diseases may be amenable to such diagnosis, treatment or prevention. Moreover, natural functions, such as embryo implantation or spermatogenesis, may be suppressed or controlled for use in birth control by molecules of the present invention.

Molecules expressed in the uterus, testis or prostate, such as zsig87 polypeptide, and which may modulate hormones, hormone receptors, growth factors, or cell-cell interactions, of the reproductive cascade or are involved in oocyte or ovarian development, spermatogenesis, or the like, would be useful as markers for cancer of reproductive organs and as therapeutic agents for hormone-dependent cancers, by inhibiting hormone-dependent growth and/or development of tumor cells. Human reproductive system cancers such as testicular, ovarian, uterine, cervical, and prostate cancers are common. Moreover, receptors for steroid hormones involved in the reproductive cascade are found in human tumors and tumor cell lines (breast, prostate, endometrial, ovarian, kidney, and pancreatic tumors) (Kakar et al., Mol. Cell. Endocrinol., 106:145-49, 1994; Kakar and Jennes, Cancer Letts., 98:57-62, 1995). Thus, expression of zsig87 in reproductive tissues suggests that polypeptides of the present invention would be useful in diagnostic methods for the detection and monitoring of reproductive cancers.

Diagnostic methods of the present invention involve the detection of zsig87 polypeptides in the serum or tissue biopsy of a patient undergoing analysis of reproductive function or evaluation for possible reproductive cancers, e.g., uterine, testicular or prostate cancer, or other cancers such as lymphomas, leukemias, colon, and mast cell cancers. Such polypeptides can be detected using immunoassay techniques and antibodies, described herein, that are capable of recognizing zsig87 polypeptide epitopes. More specifically, the present invention contemplates methods for detecting zsig87 polypeptides comprising:

exposing a test sample potentially containing zsig87 polypeptides to an antibody attached to a solid support, wherein said antibody binds to a first epitope of a zsig87 polypeptide;

washing the immobilized antibody-polypeptide to remove unbound contaminants;

exposing the immobilized antibody-polypeptide to a second antibody directed to a second epitope of a zsig87 polypeptide, wherein the second antibody is associated with a detectable label; and

detecting the detectable label. Altered levels of zsig87 polypeptides in a test sample, such as serum sweat, saliva, biopsy, and the like, can be monitored as an indication of reproductive function or of reproductive cancer or disease, when compared against a normal control.

Additional methods using probes or primers derived, for example, from the nucleotide sequences disclosed herein can also be used to detect zsig87 expression in a patient sample, such as a blood, saliva, sweat, biopsy, tissue sample, or the like. For example, probes can be hybridized to tumor tissues and the hybridized complex detected by in situ hybridization. Zsig87 sequences can also be detected by PCR amplification using cDNA generated by reverse translation of sample mRNA as a template (PCR Primer A Laboratory Manual, Dieffenbach and Dveksler, eds., Cold Spring Harbor Press, 1995). Other diagnostic detection methods include in situ hybridization of polynucleotides or anti-zsig87 antibodies, and other histologic methods known in the art. When compared with a normal control, both increases or decreases of zsig87 expression in a patient sample, relative to that of a control, can be monitored and used as an indicator or diagnostic for disease.

In addition, polypeptides of the present invention can be used for their ability to modify inflammation. Methods to assess proinflammatory or anti-inflammatory qualities of zsig87 are known in the art. For example, suppression of cAMP production is an indication of anti-inflammatory effects of the pIgR secretory component (SC) (Nihei, Y., et al., Arch. Dermatol. Res., 287:546-552, 1995). Free SC component of the poly-IgR suppressed cAMP and inhibited ICAM and HLA-Dr induced by IFN-γ in keratinocytes. Moreover, free SC has been reported to inhibit PlA2 and is believed to act via the arachadonic acid anti-inflammatory cascade. Zsig87, likewise can exhibit similar anti-inflammatory effects, and may exert these effects in tissues in which it is expressed. For example, zsig87 is expressed in mast cells and testis, and can be useful in treatment of inflammatory bowel disease, diverticulitis, inflammation during and after intestinal and testicular surgery, and the like. In addition, zsig87, expressed in mast cells, can have other anti-inflammatory actions in heart, pelvic inflammatory disease, (PID), psoriasis, arthritis, and other inflammatory diseases.

As such, zsig87 polypeptide, or its antagonists, have potential uses in inflammatory diseases such as asthma and arthritis. For example, if zsig87 is proinflammatory antagonists would be valuable in asthma therapy or other anti-inflammatory therapies where migration of lymphocytes is damaging. Alternatively, zsig87 can have an inhibitory or competitive effect on inflammatory agents and may serve directly as an asthma therapeutic or anti-inflammatory. In addition, zsig87 can serve other important roles in lung function, for instance, bronchodilation, tissue elasticity, recruitment of lymphocytes in lung infection and damage. Assays to assess the activity of zsig87 in lung cells are discussed in Laberge, S. et al., Am. J. Respir. Cell Mol. Biol. 17:193-202, 1997; Rumsaeng, V. et al., J. Immunol., 159:2904-2910, 1997; and Schluesener, H. J. et al., J. Neurosci. Res. 44:606-611, 1996. Methods to determine proinflammatory and anti-inflammatory qualities of zsig87 or its antagonists are known in the art. Moreover, other molecular biological, immunological, and biochemical techniques known in the art and disclosed herein can be used to determine zsig87 activity and to isolate agonists and antagonists.

Moreover, based on zsig87 expression in mast cells, zsig87 may exhibit antiviral functions by inhibiting viral replication by specific signaling via it's receptor(s) on a host cell (e.g. T-cell). Zsig87 may exhibit immune cell proliferative activity, as disclosed herein, and may stimulate the immune system to fight viral infections. Moreover, zsig87 may bind CD4 or another lymphocyte receptor and exhibit antiviral effects, for example, against human immunodeficiency virus (HIV) or human T-cell lymphotropic virus (HTLV). Alternatively, zsig87 polypeptide may compete for a viral receptor or co-receptor to block viral infection. Zsig87 may be given parentally to prevent viral infection or to reduce ongoing viral replication and re-infection (Gayowski, T. et al., Transplantation 64:422-426, 1997). Thus, zsig87 may be used as an antiviral therapeutic, for example, for viral leukemias (HTLV), AIDS (HIV), or gastrointestinal viral infections caused by, for example, rotavirus, calicivirus (e.g., Norwalk Agent) and certain strains of pathogenic adenovirus.

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made. Progenitor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products, and receptors. The stage of a cell population's differentiation is monitored by identification of markers present in the cell population. Myocytes, osteoblasts, adipocytes, chrondrocytes, fibroblasts and reticular cells are believed to originate from a common mesenchymal stem cell (Owen et al., Ciba Fdn. Symp. 136:42-46, 1988). Markers for mesenchymal stem cells have not been well identified (Owen et al., J. of Cell Sci. 87:731-738, 1987), so identification is usually made at the progenitor and mature cell stages. The novel polypeptides of the present invention may be useful for studies to isolate mesenchymal stem cells and myocyte or other progenitor cells, both in vivo and ex vivo.

There is evidence to suggest that factors that stimulate specific cell types down a pathway towards terminal differentiation or dedifferentiation affect the entire cell population originating from a common precursor or stem cell. Thus, the present invention includes stimulating or inhibiting the proliferation of myocytes, smooth muscle cells, osteoblasts, adipocytes, chrondrocytes, neuronal and endothelial cells. Molecules of the present invention for example, may while stimulating proliferation or differentiation of cardiac myocytes, inhibit proliferation or differentiation of adipocytes, by virtue of the affect on their common precursor/stem cells. Thus molecules of the present invention may have use in inhibiting chondrosarcomas, atherosclerosis, restenosis and obesity.

Assays measuring differentiation include, for example, measuring cell markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989; all incorporated herein by reference). Alternatively, ZSIG87 polypeptide itself can serve as an additional cell-surface or secreted marker associated with stage-specific expression of a tissue. As such, direct measurement of ZSIG87 polypeptide, or its loss of expression in a tissue as it differentiates, can serve as a marker for differentiation of tissues.

Similarly, direct measurement of ZSIG87 polypeptide, or its loss of expression in a tissue can be determined in a tissue or cells as they undergo tumor progression. Increases in invasiveness and motility of cells, or the gain or loss of expression of ZSIG87 in a pre-cancerous or cancerous condition, in comparison to normal tissue, can serve as a diagnostic for transformation, invasion and metastasis in tumor progression. As such, knowledge of a tumor's stage of progression or metastasis will aid the physician in choosing the most proper therapy, or aggressiveness of treatment, for a given individual cancer patient. Methods of measuring gain and loss of expression (of either mRNA or protein) are well known in the art and described herein and can be applied to ZSIG87 expression. For example, appearance or disappearance of polypeptides that regulate cell motility can be used to aid diagnosis and prognosis of prostate cancer (Banyard, J. and Zetter, B. R., Cancer and Metast. Rev. 17:449-458, 1999). As an effector of cell motility, ZSIG87 gain or loss of expression may serve as a diagnostic testicular, leukemic and other cancers.

Methods of measuring gain and loss of expression (of either mRNA or protein) are well known in the art and described herein and can be applied to ZSIG87 expression. For example, appearance or disappearance of polypeptides that regulate cell motility can be used to aid diagnosis and prognosis of prostate cancer (Banyard, J. and Zetter, B. R., Cancer and Metast. Rev. 17:449-458, 1999). As an effector of cell motility, or as a testis-specific and mast cell-specific marker, ZSIG87 gain or loss of expression may serve as a diagnostic for testicular cancer, mast cell and other cancers. Moreover, analogous to the prostate specific antigen (PSA), as a naturally-expressed testicular marker, increased levels of ZSIG87 polypeptides, or anti-ZSIG87 antibodies in a patient, relative to a normal control can be indicative of testicular, mast cell, leukemia, colon, CNS cancers and diseases, such as breast, non-small cell lung, prostate, renal, and ovarian cancers (See, e.g., Mulders, TMT, et al., Eur. J. Surgical Oncol. 16:37-41, 1990). Moreover, as ZSIG87 expression appears to be restricted to specific human tissues, lack of ZSIG87 expression in those tissues or strong ZSIG87 expression in tissues where ZSIG87 is not normally expressed, would serve as a diagnostic of an abnormality in the cell or tissue type, of invasion or metastasis of cancerous testicular or mast cell tissues into non-testicular or non-mast cell tissue, and could aid a physician in directing further testing or investigation, or aid in directing therapy.

In addition, as ZSIG87 is as testis and mast cell-specific, polynucleotide probes, anti-ZSIG87 antibodies, and detection the presence of ZSIG87 polypeptides in tissue can be used to assess whether these tissues are present, for example, after surgery involving the excision of a diseased or cancerous testis or mast cells. As such, the polynucleotides, polypeptides, and antibodies of the present invention can be used as an aid to determine whether all such tissue is excised after surgery, for example, after surgery for cancer. In such instances, it is especially important to remove all potentially diseased tissue or cells to maximize recovery from the cancer, and to minimize recurrence. Preferred embodiments include fluorescent, radiolabeled, or calorimetrically labeled anti-ZSIG87 antibodies and ZSIG87 polypeptide binding partners, that can be used histologically or in situ.

Moreover, the activity and effect of zsig87 on tumor progression and metastasis can be measured in vivo. Several syngeneic mouse models have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Appropriate tumor models for our studies include the Lewis lung carcinoma (ATCC No. CRL-1642) and B16 melanoma (ATCC No. CRL-6323), amongst others. These are both commonly used tumor lines, syngeneic to the C57BL6 mouse, that are readily cultured and manipulated in vitro. Tumors resulting from implantation of either of these cell lines are capable of metastasis to the lung in C57BL6 mice. The Lewis lung carcinoma model has recently been used in mice to identify an inhibitor of angiogenesis (O'Reilly M S, et al. Cell 79: 315-328,1994). C57BL6/J mice are treated with an experimental agent either through daily injection of recombinant protein, agonist or antagonist or a one time injection of recombinant adenovirus. Three days following this treatment, 10⁵ to 10⁶ cells are implanted under the dorsal skin. Alternatively, the cells themselves may be infected with recombinant adenovirus, such as one expressing zsig87, before implantation so that the protein is synthesized at the tumor site or intracellularly, rather than systemically. The mice normally develop visible tumors within 5 days. The tumors are allowed to grow for a period of up to 3 weeks, during which time they may reach a size of 1500-1800 mm³ in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed polypeptide in question, e.g., zsig87, on the ability of the tumor to recruit vasculature and undergo metastasis can thus be assessed. In addition, aside from using adenovirus, the implanted cells can be transiently transfected with zsig87. Use of stable zsig87 transfectants as well as use of induceable promoters to activate zsig87 expression in vivo are known in the art and can be used in this system to assess zsig87 induction of metastasis. Moreover, purified zsig87 or zsig87 conditioned media can be directly injected in to this mouse model, and hence be used in this system. For general reference see, O'Reilly M S, et al. Cell 79:315-328, 1994; and Rusciano D, et al. Murine Models of Liver Metastasis. Invasion Metastasis 14:349-361, 1995.

Moreover, anti-zsig87 antibodies and binding fragments can be used for tagging and sorting cells that specifically-express Zsig87, such as testis, and mast cells, and tumor cells described herein. Such methods of cell tagging and sorting are well known in the art (see, e.g., “Molecular Biology of the Cell”, 3^{rd} Ed., Albert, B. et al. (Garland Publishing, London & New York, 1994). One of skill in the art would recognize the importance of separating cell tissue types to study cells, and the use of antibodies to separate specific cell tissue types. Basically, antibodies that bind to the surface of a cell type are coupled to various matrices such as collagen, polysaccharide beads, or plastic to form an affinity surface to which only cells recognized by the antibodies will adhere. The bound cells are then recovered by conventional techniques. Other methods involve separating cells by a fluorescence-activated cell sorter (FACS). In this technique one labels cells with antibodies that are coupled to a fluorescent dye. The labeled cells are then separated from unlabeled cells in a FACS machine. In FACS sorting individual cells traveling in single file pass through a laser beam and the fluorescence of each cell is measured. Slightly further down-stream, tiny droplets, most containing either one or no cells, are formed by a vibrating nozzle. The droplets containing a single cell are automatically give a positive or negative charge at the moment of formation, depending on whether the cell they contain is fluorescent, and then deflected by a strong electric field into an appropriate container. Such machines can select 1 cell in 1000 and sort about 5000 cells each second. This produces a uniform population of cells for cell culture.

One of skill in the art would recognize that the antibodies to the Zsig87 polypeptides of the present invention are useful, because not all tissue types express the Zsig87 polypeptides and because it is important that biologists be able to separate specific cell types for further study and/or therapeutic re-implantation into the body. This is particularly relevant in cells such as testicular and mast cells, wherein zsig87 is expressed.

Zsig87 polypeptides can also be used to prepare antibodies that bind to zsig87 epitopes, peptides or polypeptides. The zsig87 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a zsig87 polypeptide (e.g., SEQ ID NO:2). Polypeptides comprising a larger portion of a zsig87 polypeptide, i.e., from 30 to 10 residues up to the entire length of the amino acid sequence are included. Antigens or immunogenic epitopes can also include attached tags, adjuvants and carriers, as described herein. Suitable antigens include the zsig87 polypeptide encoded by SEQ ID NO:2 from amino acid number 28 (Ala) to amino acid number 84 (Gly), or a contiguous 9 to 57 amino acid fragment thereof. Other suitable antigens include motifs 1 through 4, as disclosed herein. Preferred peptides to use as antigens are hydrophilic peptides such as those predicted by one of skill in the art from a hydrophobicity plot of zsig87, such as one determined from a Hopp/Woods hydrophilicity profile based on a sliding six-residue window, with buried G, S, and T residues and exposed H, Y, and W residues ignored; or a Kyte-Doolittle hydrophilicity profile; or a Jameson-Wolf antigenic index, e.g., using DNASTAR Protean program (DNASTAR, Inc., Madison, Wis.) serve as preferred antigens. Zsig87 hydrophilic peptides include peptides comprising amino acid sequences selected from the group consisting of: (1) amino acid number 39 (Cys) to amino acid number 45 (Pro) of SEQ ID NO:2; (2) amino acid number 41 (Lys) to amino acid 47 (Leu) of SEQ ID NO:2; (3) amino acid number 68 (Lys) to amino acid number 73 (Asp) of SEQ ID NO:2; (4) amino acid number 77 (Glu) to amino acid number 82 (Arg) of SEQ ID NO:2; and (5) amino acid number 68 (Lys) to amino acid number 82 (Arg) of SEQ ID NO:2 . Antibodies from an immune response generated from inoculation with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated by inoculating a variety of warm-blooded animals such as horses, cows, goats, sheep, dogs, chickens, rabbits, mice, and rats with a zsig87 polypeptide or a fragment thereof. The immunogenicity of a zsig87 polypeptide may be increased through the use of an adjuvant, such as alum (aluminum hydroxide) or Freund's complete or incomplete adjuvant. Polypeptides useful for immunization also include fusion polypeptides, such as fusions of zsig87 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Moreover, human antibodies can be produced in transgenic, non-human animals that have been engineered to contain human immunoglobulin genes as disclosed in WIPO Publication WO 98/24893. It is preferred that the endogenous immunoglobulin genes in these animals be inactivated or eliminated, such as by homologous recombination.

Alternative techniques for generating or selecting antibodies useful herein include in vitro exposure of lymphocytes to zsig87 protein or peptide, and selection of antibody display libraries in phage or similar vectors (for instance, through use of immobilized or labeled zsig87 protein or peptide). Genes encoding polypeptides having potential zsig87 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the sequences disclosed herein to identify proteins which bind to zsig87. These “binding polypeptides” which interact with zsig87 polypeptides can be used for tagging cells; for isolating homolog polypeptides by affinity purification; they can be directly or indirectly conjugated to drugs, toxins, radionuclides and the like. These binding polypeptides can also be used in analytical methods such as for screening expression libraries and neutralizing activity, e.g., for blocking interaction between ligand and receptor, or viral binding to a receptor. The binding polypeptides can also be used for diagnostic assays for determining circulating levels of zsig87 sequences disclosed herein to identify proteins which bind to zsig87. These “binding polypeptides” which interact with zsig87 polypeptides can be used for tagging cells; for isolating homolog polypeptides by affinity purification; they can be directly or indirectly conjugated to drugs, toxins, radionuclides and the like. These binding polypeptides can also be used in analytical methods such as for screening expression libraries and neutralizing activity, e.g., for blocking interaction between ligand and receptor, or viral binding to a receptor. The binding polypeptides can also be used for diagnostic assays for determining circulating levels of zsig87 polypeptides; for detecting or quantitating soluble zsig87 polypeptides as marker of underlying pathology or disease. These binding polypeptides can also act as zsig87 “antagonists” to block zsig87 binding and signal transduction in vitro and in vivo. These anti-zsig87 binding polypeptides would be useful for inhibiting zsig87 activity or protein-binding.

Antibodies are considered to be specifically binding if: 1) they exhibit a threshold level of binding activity, and 2) they do not significantly cross-react with known related polypeptide molecules. A threshold level of binding is determined if anti-zsig87 antibodies herein bind to a zsig87 polypeptide, peptide or epitope with an affinity at least 10-fold greater than the binding affinity to control (non-zsig87) polypeptide. It is preferred that the antibodies exhibit a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, G., Ann. NY Acad. Sci. 51: 660-672, 1949).

Whether anti-zsig87 antibodies do not significantly cross-react with known related polypeptide molecules is shown, for example, by the antibody detecting zsig87 polypeptide but not known related polypeptides using a standard Western blot analysis (Ausubel et al., ibid.). Examples of known related polypeptides are those disclosed in the prior art, such as known orthologs, and paralogs, and similar known members of a protein family. Screening can also be done using non-human zsig87, and zsig87 mutant polypeptides. Moreover, antibodies can be “screened against” known related polypeptides, to isolate a population that specifically binds to the zsig87 polypeptides. For example, antibodies raised to zsig87 are adsorbed to related polypeptides adhered to insoluble matrix; antibodies specific to zsig87 will flow through the matrix under the proper buffer conditions. Screening allows isolation of polyclonal and monoclonal antibodies non-crossreactive to known closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art. See, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984. Specifically binding anti-zsig87 antibodies can be detected by a number of methods in the art, and disclosed below.

A variety of assays known to those skilled in the art can be utilized to detect antibodies which bind to zsig87 proteins or polypeptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmunoprecipitation, enzyme-linked immunosorbent assay (ELISA), dot blot or Western blot assay, inhibition or competition assay, and sandwich assay. In addition, antibodies can be screened for binding to wild-type versus mutant zsig87 protein or polypeptide.

Antibodies to zsig87 and zsig87 binding polypeptides described herein may be used for tagging cells that express zsig87; for isolating zsig87 by affinity purification; for diagnostic assays for determining circulating levels of zsig87 polypeptides; for detecting or quantitating soluble zsig87 as marker of underlying pathology or disease; in analytical methods employing FACS; for screening expression libraries; for generating anti-idiotypic antibodies; for detecting or quantitating soluble zsig87 polypeptides as marker of underlying pathology or disease. These antibodies and binding polypeptides can also act as zsig87 “antagonists” to block zsig87 binding and signal transduction in vitro and in vivo. These anti-zsig87 binding polypeptides would be useful for inhibiting zsig87 activity or protein-binding.

Antibodies to zsig87 may be used for tagging cells that express zsig87; for isolating zsig87 by affinity purification; for diagnostic assays for determining circulating levels of zsig87 polypeptides; for detecting or quantitating soluble zsig87 as marker of underlying pathology or disease; in analytical methods employing FACS; for screening expression libraries; for generating anti-idiotypic antibodies; and as neutralizing antibodies or as antagonists to block zsig87 activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to zsig87 or fragments thereof may be used in vitro to detect denatured zsig87 or fragments thereof in assays, for example, Western Blots or other assays known in the art.

Antibodies or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, zsig87 polypeptides or anti-zsig87 antibodies, or bioactive fragments or portions thereof, can be coupled to detectable or cytotoxic molecules and delivered to a mammal having cells, tissues or organs that express the anticomplementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion. For these purposes, biotin/streptavidin is an exemplary complementary/anticomplementary pair.

In another embodiment, polypeptide-toxin fusion proteins or antibody-toxin fusion proteins can be used for targeted cell or tissue inhibition or ablation (for instance, to treat cancer cells or tissues). Alternatively, if the polypeptide has multiple functional domains (i.e., an activation domain or a ligand binding domain, plus a targeting domain), a fusion protein including only the targeting domain may be suitable for directing a detectable molecule, a cytotoxic molecule or a complementary molecule to a cell or tissue type of interest. In instances where the domain only fusion protein includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/cytotoxic molecule conjugates.

In another embodiment, zsig87-cytokine fusion proteins or antibody-cytokine fusion proteins can be used for enhancing in vivo killing of target tissues (for example, leukemia, testis, mast cell, CNS, colon, blood and bone marrow cancers), if the zsig87 polypeptide or anti-zsig87 antibody targets the hyperproliferative blood or bone marrow cell (See, generally, Hornick et al., Blood 89:4437-47, 1997). They described fusion proteins enable targeting of a cytokine to a desired site of action, thereby providing an elevated local concentration of cytokine. Suitable zsig87 polypeptides or anti-zsig87 antibodies target an undesirable cell or tissue (i.e., a tumor or a leukemia), and the fused cytokine mediated improved target cell lysis by effector cells. Suitable cytokines for this purpose include interleukin 2 and granulocyte-macrophage colony-stimulating factor (GM-CSF), for instance.

In yet another embodiment, if the zsig87 polypeptide or anti-zsig87 antibody targets vascular cells or tissues, such polypeptide or antibody may be conjugated with a radionuclide, and particularly with a beta-emitting radionuclide, to reduce restenosis. Such therapeutic approach poses less danger to clinicians who administer the radioactive therapy. For instance, iridium-192 impregnated ribbons placed into stented vessels of patients until the required radiation dose was delivered showed decreased tissue growth in the vessel and greater luminal diameter than the control group, which received placebo ribbons. Further, revascularisation and stent thrombosis were significantly lower in the treatment group. Similar results are predicted with targeting of a bioactive conjugate containing a radionuclide, as described herein.

The bioactive polypeptide or antibody conjugates described herein can be delivered intravenously, intraarterially or intraductally, or may be introduced locally at the intended site of action.

Molecules of the present invention can be used to identify and isolate receptors that bind zsig87. For example, proteins and peptides of the present invention can be immobilized on a column and membrane preparations run over the column (Immobilized Affinity Ligand Techniques, Hermanson et al., eds., Academic Press, San Diego, Calif., 1992, pp.195-202). Proteins and peptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62:483-514, 1993 and Fedan et al., Biochem. Pharmacol. 33:1167-80, 1984) and specific cell-surface proteins can be identified.

The molecules of the present invention will be useful for treating diabetes, and pancreatic cancer. The polypeptides, nucleic acid and/or antibodies of the present invention can be used in treatment of disorders and complications associated with these diseases. The molecules of the present invention can be used to modulate insulin, glucagon, and the like, or to treat or prevent development of pathological conditions in such diverse tissue as pancreas and other endocrine tissues. In particular, certain syndromes and diseases may be amenable to such diagnosis, treatment or prevention.

Polynucleotides encoding zsig87 polypeptides are useful within gene therapy applications where it is desired to increase or inhibit zsig87 activity. If a mammal has a mutated or absent zsig87 gene, the zsig87 gene can be introduced into the cells of the mammal. In one embodiment, a gene encoding a zsig87 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a zsig87 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Patent Publication No. WO 95/07358, published Mar. 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

It is possible to remove the target cells from the body; to introduce the vector as a naked DNA plasmid; and then to re-implant the transformed cells into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Antisense methodology can be used to inhibit zsig87 gene transcription, such as to inhibit cell proliferation in vivo. Polynucleotides that are complementary to a segment of a zsig87-encoding polynucleotide (e.g., a polynucleotide as set froth in SEQ ID NO:1) are designed to bind to zsig87-encoding mRNA and to inhibit translation of such mRNA. Such antisense polynucleotides are used to inhibit expression of zsig87 polypeptide-encoding genes in cell culture or in a subject.

The present invention also provides reagents which will find use in diagnostic applications. For example, the zsig87 gene, a probe comprising zsig87 DNA or RNA or a subsequence thereof can be used to determine if the zsig87 gene is present on a chromosome or if a mutation has occurred. Detectable chromosomal aberrations at the zsig87 gene locus include, but are not limited to, aneuploidy, loss of heterogeneity (LOH), translocations, gene copy number changes, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995).

Radiation hybrid mapping is a somatic cell genetic technique developed for constructing high-resolution, contiguous maps of mammalian chromosomes (Cox et al., Science 250:245-50, 1990). Partial or full knowledge of a gene's sequence allows one to design PCR primers suitable for use with chromosomal radiation hybrid mapping panels. Radiation hybrid mapping panels are commercially available which cover the entire human genome, such as the Stanford G3 RH Panel and the GeneBridge 4 RH Panel (Research Genetics, Inc., Huntsville, Ala.). These panels enable rapid, PCR-based chromosomal localizations and ordering of genes, sequence-tagged sites (STSs), and other nonpolymorphic and polymorphic markers within a region of interest. This includes establishing directly proportional physical distances between newly discovered genes of interest and previously mapped markers.

The precise knowledge of a gene's position can be useful for a number of purposes, including: 1) determining if a sequence is part of an existing contig and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which may aid in determining what function a particular gene might have.

Sequence tagged sites (STSs) can also be used independently for chromosomal localization. An STS is a DNA sequence that is unique in the human genome and can be used as a reference point for a particular chromosome or region of a chromosome. An STS is defined by a pair of oligonucleotide primers that are used in a polymerase chain reaction to specifically detect this site in the presence of all other genomic sequences. Since STSs are based solely on DNA sequence they can be completely described within an electronic database, for example, Database of Sequence Tagged Sites (dbSTS), GenBank, (National Center for Biological Information, National Institutes of Health, Bethesda, Md. http://www.ncbi.nlm.nih.gov), and can be searched with a gene sequence of interest for the mapping data contained within these short genomic landmark STS sequences.

The zsig87 gene is located at the 20q11.1-11.22 region of chromosome 20. There is evidence for cancer resulting from chromosomal abnormalities within in the 20q11.1-11.22 region: in malignant myeloid disorders, deletion in the long arm of chromosome 20 at 20q is evident in 95% of myeloid leukemias, with most deletions between 20q11.2-20q12 (Roulston, D et al., Blood 82:3424-3429, 1993); myeloid leukemia-related translocations involving MTGR gene at 20q11.2-q11.3 (Kitabayashi, I et al., Molec. Cell. Biol. 18:846-858, 1998); moreover, the c-src oncogene is located within the 20q breakpoint region, in 20q11.2, and increased expression is found in metastatic colon cancers. It is well known in the art that chromosome gain, or increases in copy number of a gene can result in increased expression of genes, such as c-crc within the gene region. As zsig87 is localized within this critical chromosomal region, polynucleotide probes can be used to detect chromosomal abnormalities, such as translocation, breakage, LOH, and gain associated with this region involved in myeloid cancer susceptibility, formation, or metastasis. Moreover, zsig87 is expressed in lymphoid tumor tissue and mast cells. Because there is abundant evidence for cancer resulting from mutations in the 20q11.1-11.22 region, and zsig87 also maps to this chromosomal locus, mutations in zsig87 may also be directly involved in or associated with cancers, such as lymphoid and myeloid cancers, mast cell, testicular, colon cancer or other tumors.

Moreover, zsig87 polynucleotide probes can be used to detect abnormalities or genotypes associated with some forms of Graves disease which maps to the 20q11-2-q13 region of chromosome 20 (Pearce, S et al., Am. J. Hum. Genet. 65:1462-1465, 1999; and Tomer, S et al., Am. J. Hum. Genet. 63:1749-1756, 1998). A diagnostic could assist physicians in determining the type of Graves disease and appropriate associated therapy, or assistance in genetic counseling. As such, the inventive anti-zsig87 antibodies, polynucleotides, and polypeptides can be used for the detection of zsig87 polypeptide, mRNA or anti-zsig87 antibodies, thus serving as markers and be directly used for detecting or diagnosing Graves disease or cancers, as described herein, using methods known in the art and described herein. Further, zsig87 polynucleotide probes can be used to detect abnormalities or genotypes associated with chromosome 20q11.1-11.22 deletions and translocations associated with human diseases, such as deletion, loss of heterozygosity, or translocation within 20q11.1-11.22, which are expected to be involved in chromosome rearrangements in malignancy. Similarly, zsig87 polynucleotide probes can be used to detect abnormalities or genotypes associated with chromosome 20q11.1-11.22 trisomy and chromosome loss. One of skill in the art would recognize that 20q11.1-11.22 chromosomal aberrations such as loss of heterogeneity (LOH), trisomy, rearrangements and translocations are common in several human cancers, and as such zsig87 polynucleotide probes would be useful in diagnosing and detecting such cancerous tissues and genomic aberrations associated therewith. Moreover, amongst other genetic loci, those for neuronatin (20q11.2-q12) and others manifest themselves in human disease states as well as map to this region of the human genome. See the Online Mendellian Inheritance of Man (OMIM™, National Center for Biotechnology Information, National Library of Medicine. Bethesda, Md.) gene map, and references therein, for this region of chromosome 20 on a publicly available WWW server (http://www3.ncbi.nlm.nih.gov/htbin-post/Omim/getmap?chromosome=20q11, and surrounding regions). All of these serve as possible candidate genes for an inheritable disease which show linkage to the same chromosomal region as the zsig87 gene. Thus, zsig87 polynucleotide probes can be used to detect abnormalities or genotypes associated with these defects.

Similarly, defects in the zsig87 gene itself may result in a heritable human disease state. The zsig87 gene (20q11.1-11.22) is located at a chromosomal region involved in human disease, and near an oncogene as discussed above, suggesting that this chromosomal region is commonly regulated. As, zsig87 is a ligand in a chromosomal hot spot for aberrations involved in numerous cancers and is shown to be expressed in testis, mast cells, leukemia and other cancer cells, the molecules of the present invention could also be directly involved in cancer formation or metastasis. As the zsig87 gene is located at the 20q11.1-11.22 region zsig87, polynucleotide probes can be used to detect chromosome 20q11.1-11.22 loss, trisomy, duplication or translocation associated with human diseases, such as mammary tumor tissue, breast tumor and diseased breast tissues, liver, small intestine, bone, brain or other cancers, or diseases. Moreover, molecules of the present invention, such as the polypeptides, antagonists, agonists, polynucleotides and antibodies of the present invention would aid in the detection, diagnosis prevention, and treatment associated with a zsig87 genetic defect.

A diagnostic could assist physicians in determining the type of disease and appropriate associated therapy, or assistance in genetic counseling. As such, the inventive anti-zsig87 antibodies, polynucleotides, and polypeptides can be used for the detection of zsig87 polypeptide, mRNA or anti-zsig87 antibodies, thus serving as markers and be directly used for detecting or genetic diseases or cancers, as described herein, using methods known in the art and described herein. Further, zsig87 polynucleotide probes can be used to detect abnormalities or genotypes associated with chromosome 20q11.1-11.22 deletions and translocations associated with human diseases, other translocations involved with malignant progression of tumors or other 20q11.1-11.22 mutations, which are expected to be involved in chromosome rearrangements in malignancy; or in other cancers, or in spontaneous abortion. Similarly, zsig87 polynucleotide probes can be used to detect abnormalities or genotypes associated with chromosome 20q11.1-11.22 trisomy and chromosome loss associated with human diseases. Thus, zsig87 polynucleotide probes can be used to detect abnormalities or genotypes associated with these defects.

As discussed above, defects in the zsig87 gene itself may result in a heritable human disease state. Molecules of the present invention, such as the polypeptides, antagonists, agonists, polynucleotides and antibodies of the present invention would aid in the detection, diagnosis prevention, and treatment associated with a zsig87 genetic defect. In addition, zsig87 polynucleotide probes can be used to detect allelic differences between diseased or non-diseased individuals at the zsig87 chromosomal locus. As such, the zsig87 sequences can be used as diagnostics in forensic DNA profiling. Such profiling can be applied to commercial animals as well for use in breeding programs.

In general, the diagnostic methods used in genetic linkage analysis, to detect a genetic abnormality or aberration in a patient, are known in the art. Analytical probes will be generally at least 20 nt in length, although somewhat shorter probes can be used (e.g., 14-17 nt). PCR primers are at least 5 nt in length, preferably 15 or more, more preferably 20-30 nt. For gross analysis of genes, or chromosomal DNA, a zsig87 polynucleotide probe may comprise an entire exon or more. Exons are readily determined by one of skill in the art by comparing zsig87 sequences (SEQ ID NO:1) with the human genomic DNA for zsig87 (Genbank Accession No. HS1018D12). In general, the diagnostic methods used in genetic linkage analysis, to detect a genetic abnormality or aberration in a patient, are known in the art. Most diagnostic methods comprise the steps of (a) obtaining a genetic sample from a potentially diseased patient, diseased patient or potential non-diseased carrier of a recessive disease allele; (b) producing a first reaction product by incubating the genetic sample with a ZSMF16 polynucleotide probe wherein the polynucleotide will hybridize to complementary polynucleotide sequence, such as in RFLP analysis or by incubating the genetic sample with sense and antisense primers in a PCR reaction under appropriate PCR reaction conditions; (iii) Visualizing the first reaction product by gel electrophoresis and/or other known method such as visualizing the first reaction product with a ZSMF16 polynucleotide probe wherein the polynucleotide will hybridize to the complementary polynucleotide sequence of the first reaction; and (iv) comparing the visualized first reaction product to a second control reaction product of a genetic sample from wild type patient. A difference between the first reaction product and the control reaction product is indicative of a genetic abnormality in the diseased or potentially diseased patient, or the presence of a heterozygous recessive carrier phenotype for a non-diseased patient, or the presence of a genetic defect in a tumor from a diseased patient, or the presence of a genetic abnormality in a fetus or pre-implantation embryo. For example, a difference in restriction fragment pattern, length of PCR products, length of repetitive sequences at the ZSIG87 genetic locus, and the like, are indicative of a genetic abnormality, genetic aberration, or allelic difference in comparison to the normal wild type control. Controls can be from unaffected family members, or unrelated individuals, depending on the test and availability of samples. Genetic samples for use within the present invention include genomic DNA, mRNA, and cDNA isolated form any tissue or other biological sample from a patient, such as but not limited to, blood, saliva, semen, embryonic cells, amniotic fluid, and the like. The polynucleotide probe or primer can be RNA or DNA, and will comprise a portion of SEQ ID NO:1, the complement of SEQ ID NO:1, or an RNA equivalent thereof. Such methods of showing genetic linkage analysis to human disease phenotypes are well known in the art. For reference to PCR based methods in diagnostics see see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)).

Aberrations associated with the ZSIG87 locus can be detected using nucleic acid molecules of the present invention by employing standard methods for direct mutation analysis, such as restriction fragment length polymorphism analysis, short tandem repeat analysis employing PCR techniques, amplification-refractory mutation system analysis, single-strand conformation polymorphism detection, RNase cleavage methods, denaturing gradient gel electrophoresis, fluorescence-assisted mismatch analysis, and other genetic analysis techniques known in the art (see, for example, Mathew (ed.), Protocols in Human Molecular Genetics (Humana Press, Inc. 1991), Marian, Chest 108:255 (1995), Coleman and Tsongalis, Molecular Diagnostics (Human Press, Inc. 1996), Elles (ed.) Molecular Diagnosis of Genetic Diseases (Humana Press, Inc. 1996), Landegren (ed.), Laboratory Protocols for Mutation Detection (Oxford University Press 1996), Birren et al. (eds.), Genome Analysis, Vol. 2: Detecting Genes (Cold Spring Harbor Laboratory Press 1998), Dracopoli et al. (eds.), Current Protocols in Human Genetics (John Wiley & Sons 1998), and Richards and Ward, “Molecular Diagnostic Testing,” in Principles of Molecular Medicine, pages 83-88 (Humana Press, Inc. 1998)). Direct analysis of an ZSIG87 gene for a mutation can be performed using a subject's genomic DNA. Methods for amplifying genomic DNA, obtained for example from peripheral blood lymphocytes, are well-known to those of skill in the art (see, for example, Dracopoli et al. (eds.), Current Protocols in Human Genetics, at pages 7.1.6 to 7.1.7 (John Wiley & Sons 1998)).

Mice engineered to express the zsig87 gene, referred to as “transgenic mice,” and mice that exhibit a complete absence of zsig87 gene function, referred to as “knockout mice,” may also be generated (Snouwaert et al., Science 257:1083, 1992; Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express zsig87, either ubiquitously or under a tissue-specific or tissue-restricted promoter can be used to ask whether over-expression causes a phenotype. For example, over-expression of a wild-type zsig87 polypeptide, polypeptide fragment or a mutant thereof may alter normal cellular processes, resulting in a phenotype that identifies a tissue in which zsig87 expression is functionally relevant and may indicate a therapeutic target for the zsig87, its agonists or antagonists. For example, a preferred transgenic mouse to engineer is one that over-expresses the zsig87 mature polypeptide (approximately amino acids 28 (Ala) to 84 (Gly) of SEQ ID NO:2). Moreover, such over-expression may result in a phenotype that shows similarity with human diseases. Similarly, knockout zsig87 mice can be used to determine where zsig87 is absolutely required in vivo. The phenotype of knockout mice is predictive of the in vivo effects of that a zsig87 antagonist, such as those described herein, may have. The human zsig87 cDNA can be used to isolate murine zsig87 mRNA, cDNA and genomic DNA, which are subsequently used to generate knockout mice. These transgenic and knockout mice may be employed to study the zsig87 gene and the protein encoded thereby in an in vivo system, and can be used as in vivo models for corresponding human or animal diseases (such as those in commercially viable animal populations). The mouse models of the present invention are particularly relevant as tumor models for the study of cancer biology and progression. Such models are useful in the development and efficacy of therapeutic molecules used in human cancers. Because increases in zsig87 expression, as well as decreases in zsig87 expression are associated with specific human cancers, both transgenic mice and knockout mice would serve as useful animal models for cancer. Moreover, in a preferred embodiment, zsig87 transgenic mouse can serve as an animal model for certain tissue-specific tumors particularly colon cancer, CNS cancer, or leukemia. Moreover, transgenic mice expression of zsig87 antisense polynucleotides or ribozymes directed against zsig87, described herein, can be used analogously to transgenic mice described above.

For pharmaceutical use, the proteins of the present invention are formulated for parenteral, particularly intravenous or subcutaneous, delivery according to conventional methods. Intravenous administration will be by bolus injection or infusion over a typical period of one to several hours. In general, pharmaceutical formulations will include a zsig87 protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Therapeutic doses will generally be in the range of 0.1 to 100 ?g/kg of patient weight per day, preferably 0.5-20 g/kg per day, with the exact dose determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years.

The invention is further illustrated by the following non-limiting examples.

### EXAMPLES

### Example 1

### Identification of zsig87

A. Using an EST Sequence to Obtain Full-length zsig87

Scanning of translated testis, and mast cell library DNA databases using a signal trap as a query resulted in identification of expressed sequence tag (EST) sequences that were found to be homologous to a human secretory signal sequence.

Confirmation of the EST sequence was made by sequence analyses of the cDNA from which the EST originated. This cDNA was contained in a plasmid and was sequenced using standard methods to complete the double stranded sequence of this clone. The resulting 456 bp sequence is disclosed in SEQ ID NO:1. The novel secreted polypeptide was designated ZSIG87 and is disclosed in SEQ ID NO:2.

### Example 2

### Tissue Distribution

Human Multiple Tissue Northern Blots (MTN I, MTN II, and MTN III; Clontech) are probed to determine the tissue distribution of human ZSMF16 expression. A probe is amplified from, for example, a testis or mast cell derived Marathon™-ready cDNA library (Clontech). Oligonucleotide primers are designed based on the EST sequence or cDNA sequence (SEQ ID NO:1; Example 1). The Marathon™-ready cDNA library is prepared according to manufacturer's instructions (Marathon™ cDNA Amplification Kit; Clontech) using human retina poly A+RNA (Clontech). The probe is amplified in a polymerase chain reaction under reaction conditions, for example, as follows: 1 cycle at 94?C for 1 minute; 35 cycles of 94?C for 30 seconds and 68?C for 1 minute 30 seconds; followed by 1 cycle at 72?C for 10 minutes; followed by a 4?C soak. The resulting DNA fragment is electrophoresed on an approximately 2% low melt agarose gel (SEA PLAQUE GTG low melt agarose, FMC Corp., Rockland, Me.), the fragment is purified using the QIAquick™ method (Qiagen, Chatsworth, Calif.), and the sequence is confirmed by sequence analysis.

The probe is radioactively labeled and purified as described herein using methods known in the art. ExpressHyb™ (Clontech) solution, or similar hybridization solution, is used for prehybridization and as a hybridizing solution for the Northern blots. Hybridization takes place overnight at 65° C. using about 1.0×10⁶ cpm/ml of labeled probe. The blots are then washed about 4 times at room temperature in 2×SSC, 0.05% SDS followed by about 2 washes at 50° C. in 0.1×SSC, 0.01% SDS for about 20 minutes each. A transcript of approximately 0.240-1 kb should be seen in tissues that express the ZSIG87 mRNA.

Additional analysis can be carried out on Northern blots made with poly(A) RNA from the human vascular cell lines HUVEC (human umbilical vein endothelial cells; Cascade Biologics, Inc., Portland, Oreg.), HPAEC (human pulmonary artery endothelial cells; Cascade Biologics, Inc.), HAEC (human aortic endothelial cells; Cascade Biologics, Inc.), AoSMC (aortic smooth muscle cells; Clonetics, San Diego, Calif.), UASMC (umbilical artery smooth muscle cells; Clonetics), HISM (human intestinal smooth muscle cells; ATCC CRL 7130), SK-5 (human dermal fibroblast cells; obtained from Dr. Russell Ross, University of Washington, Seattle, Wash.), NHLF (normal human lung fibroblast cells; Clonetics), and NHDF-NEO (normal human dermal fibroblast-neonatal cells; Clonetics). The probe is prepared and labeled and prehybridization and hybridization were carried out essentially as disclosed above. The blots are then washed at about 50° C. in 0.1×SSC, 0.05% SDS. A transcript of approximately 0.240-1 kb should be seen in those cells that express the ZSIG87 mRNA.

Additional analysis can be carried out on Northern blots made with poly(A) RNA from K-562 cells (erythroid, ATCC CCL 243), HUT78 cells (T cell, ATCC TIB-161), Jurkat cells (T cell), DAUDI (Burkitt's human lymphoma, Clontech, Palo Alto, Calif.), RAJI (Burkitt's human lymphoma, Clontech) and HL60 (Monocyte). The probe preparation and hybridization are carried out as above. A transcript of approximately 0.240-1 kb should be seen in those cells that express the ZSIG87 mRNA.

Additional analysis can be carried out on Northern blots made with poly (A) RNA from CD4⁺, CD8⁺, CD19⁺ and mixed lymphocyte reaction cells (CellPro, Bothell, Wash.) using probes and hybridization conditions described above. A transcript of approximately 0.240-1 kb should be seen in those cells that express the ZSIG87 mRNA.

Additional analysis can be carried out on Human Brain Multiple Tissue Northern Blots II and III (Clontech) using the probe and hybridization conditions described above. A transcript of approximately 0.240-1 kb should be seen in those cells that express the ZSIG87 mRNA.

Moreover a Dot Blot is also performed using Human RNA Master Blots? (Clontech). The methods and conditions for the Dot Blot were the same as for the Multiple Tissue Blots disclosed above. Again, a signal is present for those tissues that express the ZSIG87 mRNA.

### Example 3

### Chromosomal Assignment and Placement of ZSIG87

ZSIG87 is mapped to a human chromosome using the commercially available GeneBridge 4 Radiation Hybrid Panel (Research Genetics, Inc., Huntsville, Ala.). The GeneBridge 4 Radiation Hybrid Panel contains DNAs from each of 93 radiation hybrid clones, plus two control DNAs (the HFL donor and the A23 recipient). A publicly available WWW server (http://www-genome.wi.mit.edu/cgi-bin/contig/rhmapper.pl) allows mapping relative to the Whitehead Institute/MIT Center for Genome Research's radiation hybrid map of the human genome (the “WICGR” radiation hybrid map) which was constructed with the GeneBridge 4 Radiation Hybrid Panel.

For the mapping of ZSIG87 with the GeneBridge 4 RH Panel, 20 μl reactions are set up in a 96-well microtiter plate (Stratagene, La Jolla, Calif.) and used in a RoboCycler Gradient 96 thermal cycler (Stratagene). Each of the 95 PCR reactions consist of 2 μl 10× KlenTaq PCR reaction buffer (Clontech), 1.6 μl dNTPs mix (2.5 mM each, PERKIN-ELMER, Foster City, Calif.), 1 μl sense primer, 1 μl antisense primer, 2 μl RediLoad (Research Genetics, Inc.), 0.4 μl 50× Advantage KlenTaq Polymerase Mix (Clontech), 25 ng of DNA from an individual hybrid clone or control and ddH₂O for a total volume of 20 μl. The reactions are overlaid with an equal amount of mineral oil and sealed. The PCR cycler conditions are, for example, as follows: an initial 1 cycle 5 minute denaturation at 95° C., 35 cycles of a 1 minute denaturation at 95° C., 1 minute annealing at 66° C. and 1.5 minute extension at 72° C., followed by a final 1 cycle extension of 7 minutes at 72° C. The reactions are separated by electrophoresis on a 2% agarose gel (Life Technologies, Gaithersburg, Md.).

An STS is defined by a pair of oligonucleotide primers that are used in a polymerase chain reaction, as describe above, to specifically detect this site in the presence of all other genomic sequences. Since STSs are based solely on DNA sequence they can be completely described within an electronic database, for example, Database of Sequence Tagged Sites (dbSTS), GenBank, (National Center for Biological Information, National Institutes of Health, Bethesda, Md. http://www.ncbi.nlm.nih.gov), and can be searched with a gene sequence of interest for the mapping data contained within these short genomic landmark STS sequences, or on the WICGR radiation hybrid map. Proximal and distal framework markers can be determined as well. The use of surrounding markers will position ZSIG87 in a defined region on the integrated LDB chromosome map (The Genetic Location Database, University of Southhampton, WWW server: http://cedar. genetics.soton.ac.uk/public html/).

### Example 4

### Expression of zsig87 in Cancer Tissues Using NCI60 Cancer Microarray

A. Determination of Genes having Correlated Expression with zsig87

Gene expression profile information for zsig87 was obtained from oligonucleotide and cDNA microarrays. Microarrays show the mRNA expression level of a large number of genes across a large number of cell types or cells exposed to various conditions, or cells in various replication steps, depending on the experiment. Because all of the information for all of the genes on any given microarray is obtained from the same biological experiment, and all biological experiments employing the same microarray provide results on the same set of genes, it is possible to compare the mRNA expression patterns of different genes to each other, as well as the expression pattern of a given gene in various tissues, cell lines, or cancers.

Briefly, microarray experiments are conducted by extracting the mRNA from reference tissues(s) or cell line(s) and from experimental sample tissue(s) or cell line(s). The reference mRNA is reverse transcribed to cDNA in a reaction along with a fluorescent dye label. The sample mRNA is likewise reverse transcribed to cDNA, but in the presence of a dye label with a different emission wavelength from the reference. The two cDNA samples are then mixed and hybridized to the microarray. The microarray itself has thousands of unlabeled cDNA clones covalently bound as spots (also called ‘features’) on its surface. The labeled cDNAs then bind to their respective microarray spots. If a particular gene is transcribed at a higher level in the experimental sample relative to the reference, then the spot will fluoresce to a greater degree in the experimental sample dye wavelength channel. Conversely, if the gene in the experimental sample is down regulated, then the wavelength channel of the reference dye will be stronger. Finally, the microarrays are scanned at the wavelengths of both dyes and the results for each spot are recorded and stored electronically. Large numbers of microarray experiments are typically done together using the same reference cDNA, but varying the experimental conditions, cell lines, tissues, time points, and the like.

Raw and/or processed microarray expression information was obtained from a subscription data set that was electronically downloaded. Publicly available, purchased, or in-house custom designed software can be used to analyze the microarray data (E.g., the publicly available NCI60 Cancer Microarray Project (Stanford University, Palo Alto, Calif.) world-wide-web resource http://genome-www.stanford/nci60/search.shtml). Prior to analysis, spots were examined to exclude experimental artifacts (dust spots, substrate imperfections, incomplete or uneven hybridization washes, etc.) and absorbence was adjusted to take into account background fluorescence of the microarray substrate at both wavelengths. Very weak and very strong signals beyond the linear range response of the microarray reader were likewise excluded from analysis. Analyses were typically done on the ratio of the absorbance intensities of the reference and sample wavelength channels for each spot. These absorbance ratios were normalized to log base 2. Microarray information for zsig87 was found in Ross et al. using a ‘NCI60’ microarray (Ross, DT et al., Nature Genet. 24:227-235, 2000). The reference mRNA was composed of a mixture of equal quantities of mRNA from HL-60, K562, NCI-H226, COLO 205, SNB-19, LOX-IMVI, OVCAR-3, OVCAR-4, CAKI-1, PC-3, MCF7, and Hs578T cells. See, Ross et al. supra. for details of this method.

A cDNA clone corresponding to an area adjacent to the genomic region that encodes zsig87 cDNA (and corresponding mRNA) was included on the ‘NCI60’ microarray chip set (Ross et al. supra.). This cDNA clone (IMAGE clone 221486; Incyte Pharmaceuticals, Palo Alto, Calif.) corresponds to nucleotide positions 39269 to 39828 of HS1018D12 (located within the genomic DNA encompassing zsig87). Expression corresponding to nucleotide positions 39269 to 39828 of HS1018D12 is expected to be correlated with zsig87 expression (Kelly B L, and Locksley R M, J. Immunol. 165:2982-6, 2000; Lacy D A et al., J. Immunol. 164:4569-74, 2000; Trappe R et al., Biochim. Biophys. Acta. 1446:341-51, 1999; Mesilaty-Gross S et al., Gene 231:173-86, 1999; and Duboule D, Curr. Opin. Genet. Dev. 8:514-8, 1998). This chip set contained 9702 additional cloned cDNAs. Ross et al. performed 68 hybridization experiments with this chip set against 60 cancer cell lines. The data from the NCI60 microarray was purchased through the SUTECH™ Microarray Expression Database Subscription Program from Stanford Sequencing and Technology Center's Technology Development Group, (Stanford University, Palo Alto, Calif.) (http://otl.stanford.edu/tech/sutech.html).

Analysis was done by obtaining the Pearson correlation (R) between all pairs of spots in the entire set of microarray experiments. The Pearson correlation comprises a value from 1 to −1. A value of 1 shows that the expression in the two compared spots are positively correlated (both either are increased or decreased). A value of −1 shows that the expression in the two compared spots are negatively correlated (when one goes up, the other goes down, or visa versa). A value of 0 shows that the items are not correlated over the range of experiments. Although values between 0 and 1, or 0 and −1 can be considered positively or negatively correlated respectively, in the current analysis, correlations greater than 0.55 or less than −0.55 were considered to be significant. A similar analysis was performed by Ross et al, supra. Their results were also queried electronically by the NCI60 Cancer Microarray Project (Stanford University, Palo Alto, Calif.) world-wide-web resource (http://genome-www.stanford/nci60/search.shtml). Thus genes potentially co-regulated or coexpressed with zsig87 were evaluated.

Table 5 shows the results of correlated cDNA clones in the NCI60 microarray that have a Pearson's R correlation of expression greater than 0.55 or less than −0.55 with a z87 expression or were observed in the results of Ross et al, supra. Expressed genes are indexed by their accession number, and the corresponding protein, if known, is described.

Our analysis of the data showed that zsig87 had correlated expression (Pearson's R<−0.55 or >0.55) with 9 other cDNA clones (Table 5). The clones having correlated expression with zsig87 included metabolism and an aveolar surfacant protein. For example, zsig87 was coexpressed with creatin kinase B chain, and acetyl-CoA carboxylase beta. Examination of the results obtained by our analysis, in conjunction with the results obtained from Stanford Genomic Resources (Stanford University, Palo Alto, Calif.) (http://genome-www.stanford.edu/) also reveals that zsig87 is coexpressed with FBLN1 (fibulin 1—an endocardium extracellular matrix protein), ACVRL1 (activin A receptor type II-like 1—a member of the tgf-beta receptor superfamily), and PRELP (a proline arginine-rich end leucine-rich repeat protein—part of the extracellular matrix of cartilage and lung tissue). These results show that zsig87 is co-expressed with cardiac and pulmonary matrix proteins as well as a potential growth factor receptor and metabolism proteins, reinforces the premise that zsig87 is involved in tissue and organ remodeling and growth as described herein.

TABLE 5

| Genbank | | |
|---|---|---|
| Accession No. | Pearson's R | Description |
| AA015936-AA016045 | 0.550117 | HSCKBG, creatin kinase, B chain |
| R13965-R40025 | 0.571980 | Unknown |
| H89843-H90692 | 0.581399 | Unknown |
| N44012-N34945 | 0.581410 | COA2_HUMAN, |
| | | acetyl-CoA carboxylase beta |
| H24131-H22949 | 0.591669 | Unknown |
| AA004705 | 0.598803 | HSAPC3A, apolipoprotein apoAI |
| H66213 | 0.602370 | Unknown |
| H04934-H04828 | 0.628666 | Unknown |
| W86500-W86624 | 0.634072 | AE176520, WD |
| | | repeat-containing F-box |

B. Determination of zsig87 Expression in Cell and Tissue Types, and Cancers

Zsig87 expression in the microarray hybridization data described above was also analyzed for expression in various tissue types and cancers. Table 6 shows the Ratio of expression of zsig87 relative to the reference standard. The ratio of expression is another was to view the data. For each spot on the microarray, the ratio of fluorescence of the reference and sample wavelengths is a measure of the level of induction or repression of the test sample relative to the control (Ratio=[sample fluorescence/control reference fluorescence]). If there is no change in mRNA expression level of a given gene in the control and test samples, then the ratio for the corresponding spot will be 1. If the sample expression is induced in the test sample then the ratio of fluorescence for that spot will be greater than 1; if it is repressed then the ratio will be less than 1.

The results indicated that zsig87 is up-regulated in some colon cancer and leukemia cell lines. This data also indicated a down-regulation of zsig87 in a CNS cancer cell lines. However, most colon, leukemia, prostate, CNS, renal, breast, and non small cell lung cancer cell lines generally showed mixed or weak changes in zsig87 expression relative to the control level. Zsig87 expression was highest in the HCT-15, HCC-2998 (colon cancer) cell lines, and the HL-60 and K562 (leukemia) cell lines. Zsig87 expression was lowest in the SF-539 CNS cell line. These results show that a zsig87 increase or decrease in expression is correlated with certain human cancers. As such, detection of zsig87 expression increase or decrease can be used as a diagnostic for human cancers. Moreover, in a preferred embodiment, zsig87 can serve as a marker for certain tissue-specific tumors particularly colon cancer, CNS cancer, or leukemia. Moreover, detection of zsig87 expression increase or decrease may be used as a diagnostic for other human cancers in which differential expression may be evident, such as breast, non-small cell lung, prostate, renal, and ovarian cancers. Moreover, in a preferred embodiment, zsig87 can serve as a marker for certain tissue-specific tumors Use of polynucleotides, polypeptides, and antibodies of the present invention for such diagnostic purposes are known in the art, and disclosed herein.

TABLE 6

| | | Ratio of Zsig87 |
|---|---|---|
| | | expression |
| Cell Line or Tissue | Description | to reference |
| BT-549 | breast cancer cell line | 1 |
| HS_5781 | breast cancer cell line | 1 |
| MCF7A | breast cancer cell line | 1 |
| MCF7D | breast cancer cell line | 1 |
| MCF7 | breast cancer cell line | 1 |
| MDA-MB-231 | breast cancer cell line | 1 |
| MDA-MB-435 | breast cancer cell line | 1 |
| MDA-N | breast cancer cell line | 1 |
| T-47D | breast cancer cell line | 1 |
| BC2_Lymph_Node | breast cancer lymph node metastasis | 1 |
| BC16 | breast cancer tissue biopsy | 1 |
| BC2 | breast cancer tissue biopsy | 1 |
| Normal_Breast | breast tissue biopsy, normal | 1 |
| SF-268 | CNS cancer cell line | 1 |
| SF-295 | CNS cancer cell line | 1 |
| SF-539 | CNS cancer cell line | 0.54 |
| SNB-19 | CNS cancer cell line | 1 |
| U251 | CNS cancer cell line | 1 |
| COLO205 | colon cancer cell line | 1 |
| HCC-2998 | colon cancer cell line | 1.55 |
| HCT-116 | colon cancer cell line | 1 |
| HCT-15 | colon cancer cell line | 1.77 |
| HT-29 | colon cancer cell line | 1 |
| KM12 | colon cancer cell line | 1.07 |
| SW-620 | colon cancer cell line | 0.9 |
| CCRF-CEM | leukemia cell line | 1 |
| HL-60 | leukemia cell line | 1.74 |
| K-562 | leukemia cell line | 1.42 |
| K562A | leukemia cell line | 1 |
| K562B | leukemia cell line | 1 |
| MOLT-4 | leukemia cell line | 1 |
| RPMI-8226 | leukemia cell line | 0.8 |
| SR | leukemia cell line | 1 |
| LOXLMVI | melanoma cell line | 1.02 |
| M-14 | melanoma cell line | 1 |
| MALME-3M | melanoma cell line | 1 |
| SK-MEL-28 | melanoma cell line | 1 |
| SK-MEL-2 | melanoma cell line | 1 |
| SK-MEL-5 | melanoma cell line | 1 |
| UACC-257 | melanoma cell line | 1 |
| UACC-62 | melanoma cell line | 1 |
| A549 | non-small cell lung cancer (NSCLC) cell | 0.97 |
| | line | |
| EKVX | NSCLC cell line | 1 |
| HOP-62 | NSCLC cell line | 1 |
| HOP-92 | NSCLC cell line | 1 |
| NCI-H226 | NSCLC cell line | 1 |
| NCI-H23 | NSCLC cell line | 0.82 |
| NCI-H322 | NSCLC cell line | 1.22 |
| NCI-H460 | NSCLC cell line | 1 |
| NCI-H522 | NSCLC cell line | 1 |
| IGROV1 | ovarian cancer cell line | 1.31 |
| OVCAR-3 | ovarian cancer cell line | 1 |
| OVCAR-4 | ovarian cancer cell line | 0.86 |
| OVCAR-5 | ovarian cancer cell line | 1.15 |
| OVCAR-8 | ovarian cancer cell line | 1 |
| SK-OV-3 | ovarian cancer cell line | 1 |
| DU-145 | prostate cancer cell line | 1.24 |
| PC-3 | prostate cancer cell line | 1.19 |
| 786-0 | renal cancer cell line | 1 |
| A498 | renal cancer cell line | 1 |
| ACHN | renal cancer cell line | 1.27 |
| CAKI-1 | renal cancer cell line | 1 |
| RXF-393 | renal cancer cell line | 1.36 |
| SN12C | renal cancer cell line | 1 |
| SNB-75 | renal cancer cell line | 1 |
| TK-10 | renal cancer cell line | 1 |
| UO-31 | renal cancer cell line | 1 |
| ADR-RES | unknown | 1.38 |

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polynucleotide encoding a polypeptide comprising a sequence of amino acid residues that is at least 90% identical to an amino acid sequence selected from the group consisting of:
(a) the amino acid sequence as shown in SEQ ID NO:2 from amino acid number 27 (Arg) to amino acid number 56 (Cys);
(b) the amino acid sequence as shown in SEQ ID NO:2 from amino acid number 16 (Glu) to amino acid number 84 (Pro); and
(c) the amino acid sequence as shown in SEQ ID NO:2 from amino acid number 1 (Met) to amino acid number 84 (Pro).

2. An isolated polynucleotide according to claim 1, wherein the polynucleotide is selected from the group consisting of:
(a) a polynucleotide sequence as shown in SEQ ID NO:1 from nucleotide 198 to nucleotide 287;
(b) a polynucleotide sequence as shown in SEQ ID NO:1 from nucleotide 165 to nucleotide 371;
(c) a polynucleotide sequence as shown in SEQ ID NO:1 from nucleotide 120 to nucleotide 371; and
(c) a polynucleotide sequence complementary to (a) or (b).

3. An isolated polynucleotide sequence according to claim 1, wherein the polynucleotide comprises nucleotide 1 to nucleotide 252 of SEQ ID NO:3.

4. An isolated polynucleotide according to claim 1, wherein the polypeptide comprises a sequence of amino acid residues selected from the group consisting of:
(a) the amino acid sequence as shown in SEQ ID NO:2 from amino acid number 27 (Arg) to amino acid number 56 (Cys);
(b) the amino acid sequence as shown in SEQ ID NO:2 from amino acid number 16 (Glu) to amino acid number 84 (Pro); and
(c) the amino acid sequence as shown in SEQ ID NO:2 from amino acid number 1 (Met) to amino acid number 84 (Pro).

5. The isolated polynucleotide molecule of claim 1, wherein the polynucleotide encodes a polypeptide that further comprises a cysteine motif spaced apart from N-terminus to C-terminus in a configuration represented by C-{4}-G-C-(K/R)-{2}-C-{9/13}-C-{5}-C-C,
where “C” is cysteine,
“G” is Glycine,
“(K/R)” is Lysine or Arginine,
“{#}” denotes the number of amino acid residues between the above amino acids,
“{9/13}” denotes that the number of amino acid residues between the above amino acids is 9 or 13.

6. An expression vector comprising the following operably linked elements:
a transcription promoter;
a DNA segment encoding a polypeptide comprising an amino acid sequence as shown in SEQ ID NO:2 from amino acid number 16 (Glu) to amino acid number 84 (Pro); and
a transcription terminator.

7. An expression vector according to claim 6, further comprising a secretory signal sequence operably linked to the DNA segment.

8. A cultured cell into which has been introduced an expression vector according to claim 6, wherein the cell expresses a polypeptide encoded by the DNA segment.

9. A DNA construct encoding a fusion protein, the DNA construct comprising:
a first DNA segment encoding a polypeptide comprising a sequence of amino acid residues selected from the group consisting of:
(a) the amino acid sequence of SEQ ID NO: 2 from residue number 1 (Met) to residue 15 (Glu);
(b) the amino acid sequence of SEQ ID NO: 2 from residue number 27 (Arg) to amino acid number 84 (Pro); and
(b) the amino acid sequence of SEQ ID NO: 2 from residue number 16 (Glu) to amino acid number 84 (Pro); and
at least one other DNA segment encoding an additional polypeptide,
wherein the first and other DNA segments are connected in-frame; and
encode the fusion protein.

10. A fusion protein produced by a method comprising:
culturing a host cell into which has been introduced a vector comprising the following operably linked elements:
(a) a transcriptional promoter;
(b) a DNA construct encoding a fusion protein according to claim 9; and
(c) a transcriptional terminator; and
recovering the protein encoded by the DNA segment.

11. An isolated polypeptide comprising a sequence of amino acid residues that is at least 90% identical to an amino acid sequence selected from the group consisting of:
(a) the amino acid sequence as shown in SEQ ID NO:2 from amino acid number 27 (Arg) to amino acid number 56 (Cys);
(b) the amino acid sequence as shown in SEQ ID NO:2 from amino acid number 16 (Glu) to amino acid number 84 (Pro); and
(c) the amino acid sequence as shown in SEQ ID NO:2 from amino acid number 1 (Met) to amino acid number 84 (Pro).

12. An isolated polypeptide according to claim 11, wherein the polypeptide further comprises a cysteine motif spaced apart from N-terminus to C-terminus in a configuration represented by C-{4}-G-C-(K/R)-{2}-C-{9/13}-C-{5}-C-C,
where “C” is cysteine,
“G” is Glycine,
“(K/R)” is Lysine or Arginine,
“{#}” denotes the number of amino acid residues between the above amino acids,
“{9/13 }” denotes that the number of amino acid residues between the above amino acids is 9 or 13.

13. An isolated polypeptide according to claim 11, wherein the polypeptide comprises a sequence of amino acid residues selected from the group consisting of:
(a) the amino acid sequence as shown in SEQ ID NO:2 from amino acid number 27 (Arg) to amino acid number 56 (Cys);
(b) the amino acid sequence as shown in SEQ ID NO:2 from amino acid number 16 (Glu) to amino acid number 84 (Pro); and
(c) the amino acid sequence as shown in SEQ ID NO:2 from amino acid number 1 (Met) to amino acid number 84 (Pro).

14. A method of producing a polypeptide comprising:
culturing a cell according to claim 8; and
isolating the polypeptide produced by the cell.

15. A method of producing an antibody to a polypeptide comprising the following steps in order:
inoculating an animal with a polypeptide selected from the group consisting of:
(a) a polypeptide according to claim 11;
(b) a polypeptide comprising the amino acid sequence of SEQ ID NO: 2 from residue number 16 (Glu) to amino acid number 84 (Pro);
(c) a polypeptide comprising the amino acid sequence of SEQ ID NO: 2 from residue number 27 (Arg) to amino acid number 56 (Cys);
(d) a polypeptide comprising of the amino acid sequence of SEQ ID NO: 2 from residue number 57 (Leu) to amino acid number 84 (Pro);
(e) a polypeptide consisting of amino acid number 39 (Cys) to amino acid number 45 (Pro) of SEQ ID NO:2;
(f) a polypeptide consisting of amino acid number 41 (Lys) to amino acid 47 (Leu) of SEQ ID NO:2;
(g) a polypeptide consisting of amino acid number 68 (Lys) to amino acid number 73 (Asp) of SEQ ID NO:2;
(h) a polypeptide consisting of amino acid number 77 (Glu) to amino acid number 82 (Arg) of SEQ ID NO:2; and
(i) a polypeptide consisting of amino acid number 68 (Lys) to amino acid number 82 (Arg) of SEQ ID NO:2; and
wherein the polypeptide elicits an immune response in the animal to produce the antibody; and isolating the antibody from the animal.

16. An antibody produced by the method of claim 15, which binds to a polypeptide of SEQ ID NO:2.

17. The antibody of claim 16, wherein the antibody is a monoclonal antibody.

18. An antibody which specifically binds to a polypeptide of claim 13.

19. A method for detecting a genetic abnormality in a patient, comprising:
obtaining a genetic sample from a patient;
producing a first reaction product by incubating the genetic sample with a polynucleotide comprising at least 14 contiguous nucleotides of SEQ ID NO:1 or the complement of SEQ ID NO:1, under conditions wherein said polynucleotide will hybridize to complementary polynucleotide sequence;
visualizing the first reaction product; and
comparing said first reaction product to a control reaction product from a wild type patient, wherein a difference between said first reaction product and said control reaction product is indicative of a genetic abnormality in the patient.

20. A method for detecting a cancer in a patient, comprising:
obtaining a tissue or biological sample from a patient;
incubating the tissue or biological sample with an antibody of claim 18 under conditions wherein the antibody binds to its complementary polypeptide in the tissue or biological sample;
visualizing the antibody bound in the tissue or biological sample; and
comparing levels of antibody bound in the tissue or biological sample from the patient to a normal control tissue or biological sample,
wherein an increase or decrease in the level of antibody bound to the patient tissue or biological sample relative to the normal control tissue or biological sample is indicative of a cancer in the patient.

21. A method for detecting a cancer in a patient, comprising:
obtaining a tissue or biological sample from a patient;
labeling a polynucleotide comprising at least 14 contiguous nucleotides of SEQ ID NO:1 or the complement of SEQ ID NO:1;
incubating the tissue or biological sample with under conditions wherein the polynucleotide will hybridize to complementary polynucleotide sequence;
visualizing the labeled polynucleotide in the tissue or biological sample; and
comparing the level of labeled polynucleotide hybridization in the tissue or biological sample from the patient to a normal control tissue or biological sample,
wherein an increase or decrease in the labeled polynucleotide hybridization to the patient tissue or biological sample relative to the normal control tissue or biological sample is indicative of a cancer in the patient.
